(19) United States
(12) Patent Application Publication
(10) Pub. No.: US 2006/0199183 A1
(43) Pub. Date: Sep. 7, 2006

(54) Probe biochips and methods for use thereof

(76) Inventors: Christophe Valat, Villiers Sur Marne (FR); Cecile Le Douguet, Courbevoie (FR)

Correspondence Address: NIXON & VANDERHYE, PC, 901 NORTH GLEBE ROAD, 11TH FLOOR, ARLINGTON, VA, 22203, US

(21) Appl. No.: 10/548,174

(22) PCT Filed: Mar. 11, 2004
(86) PCT No.: PCT/FR04/00586

§ 371 Date: Mar. 13, 2006

(30) Foreign Application Priority Data
Mar. 13, 2003 (FR) 0303112

(51) Int. Cl. C12Q 1/68; C07H 21/04; C12M 1/34

(52) U.S. Cl. 435/6; 435/287.2; 536/24.3

(57) ABSTRACT

The invention relates to fields of use of unlabelled polynucleotide probes able to form hairpins, the biochips comprising such probes and methods allowing use thereof. The present invention also concerns methods for designing such probes and biochips. More particularly, the invention concerns the use of such unlabelled probes and biochips for manipulating and analysing polynucleotide sequences and optionally molecules which are associated therewith. This invention further concerns methods for preparing and use such probes and biochips for analysing mutations, sequencing, detection of alternative splicing variants, gene expression analysis, analysis of allelic imbalances and loss of heterozygosity and the detection of any nucleic acid present in organisms or residues from said organisms.

The invention relates to the use of unlabeled polynucleotide probes, which can form hairpin loops, biochips comprising said probes and method for the use thereof. The invention further relates to methods for the design of said probes and biochips. More particularly the invention relates to the use of said unlabeled probes and biochips for the manipulation and the analysis of polynucleotide sequences and optionally associated molecules. Furthermore, the invention relates to the methods for the preparation and use of said probes and biochips for the analysis of mutations, sequencing, detection of alternative splicing variants, analysis of gene expression, analysis of allelic imbalances and loss of heterozygosity and the detection of any nucleic acid present in organisms or residues from said organisms.

Rapid and accurate determination of the identities and abundance of specific molecules in a sample containing many different molecules is of great interest in biological and medical fields. Many types of probes and assay systems have been created for detecting specific molecules, e.g., probes and biochips directed to detecting varying nucleic acid sequences.

One such probe for detecting specific nucleic acids is a “molecular switch” disclosed in U.S. Pat. No. 5,118,801 which works on the principle that the ends of the probe are unable to interact with each other when the centre portion of the probe hybridizes with a target sequence. Each molecular switch probe has two complementary ends, which are at least 10 nucleotides in length, and a centre portion of about 15-115 nucleotides in length. Based upon the assay conditions, the disclosed probe can have a “closed” or “open” conformation. When the probe is in the closed conformation, the ends of the probe hybridize to one another to form a “stem” with the centre portion forming a “loop.” In the open conformation, the centre portion of the probe hybridizes to a predetermined complementary target sequence for which the probe is designed. This open conformation results in the dissociation of the probe ends thereby leaving the ends unable to interact with each other. One or both of the ends in the disclosed probe contains a biologically functional nucleic acid moiety useful for selectively generating a detectable signal indicative of the hybridization of the probe with its predetermined target sequence. For example, a preferred moiety is a RNA that permits exponential replication by a RNA-directed RNA polymerase where radioactive nucleotides are integrated into the replicants.

Another probe type, commonly referred to as a “molecular beacon,” is similar to that above described and is disclosed in U.S. Pat. No. 5,925,517 (see also Tyagi et al., 1996, Nat. Biotechnol. 14:303-308; Tyagi et al., 1998, Nat. Biotechnol. 16:49-53; Matsuo T. 1998, Biochimica Biophysica Acta. 1379:178-184; Sokol et al. 1998, Proc. Natl. Acad. Sci. USA 95:11538-11543; Leone et al. 1998, Nucleic Acids Res. 26:2150-2155; Piatek et al. 1998, Nat. Biotechnol. 16:359-363; Kostrikis et al. 1998, Science 279:1228-1229; Giesendorf et al. 1998, Clin. Chem. 44:482-486; Marras et al. 1999 Genet. Anal. 14:151-156; Vet et al. 1999, Proc. Natl. Acad. Sci. USA 96:6394-6399). Molecular beacons are molecules with an internally quenched fluorophore whose fluorescence is restored when they bind to a target nucleic acid. They are designed similarly to a molecular switch so that the loop portion of the molecule is a probe sequence complementary to a target nucleic molecule and the stem is formed by the annealing of complementary “arm” sequences on the ends of the probe sequence. Attached to the end of one arm is a fluorescent moiety. Attached to the end of the other arm is a quenching moiety. In the absence of a target molecule, these two moieties are in close proximity to each other, causing the fluorescence of the fluorophore to be quenched. When the probe hybridizes with a perfectly complementary target molecule, the molecular beacon undergoes a conformational change that forces the stem apart thereby causing the fluorophore and the quencher to move away from each other. This allows to detect hybridization of the molecular beacon with its target molecule through fluorescence appearance.

Presently, biochip systems are widely used for the detection and measurement of particular substances in complex samples. In such systems, the identity and abundance of a target substance in a sample is determined by measuring the level of association of the target sequence to probes specifically designed for that target sequence. In nucleic acid biochip technologies, a set of nucleic acid probes, each of which has a defined sequence, is immobilized on a solid support in such a manner that each probe is immobilized to a predetermined region. The set of immobilized probes, the biochip, is contacted with a sample so that sequences complementary to an immobilized probe may associate, e.g., hybridize, anneal, or bind, to the probe. After removing any non-associated material, the associated sequences are detected and measured.

DNA biochip technologies have made it possible to monitor the expression levels of a large number of genetic transcripts at the same time (see, e.g., Schena et al., 1995, Science 270:467-470; Lockhart et al., 1996, Nature Biotechnology 14:1675-1680; Blanchard et al., 1996, Nature Biotechnology 14:1649; Ashby et al., U.S. Pat. No. 5,569,588 issued Oct. 29, 1996). Biochip technology has also been used to sequence, fingerprint, and map biological macromolecules (U.S. Pat. No. 6,270,961 issued Aug. 7, 2001; U.S. Pat. No. 6,025,136 issued Feb. 15, 2000; U.S. Pat. No. 6,018,041 issued Jan. 25, 2000; U.S. Pat. No. 5,871,928 issued Feb. 16, 1999; U.S. Pat. No. 5,695,940 issued Dec. 9, 1997). There is two main formats of DNA biochips. In one of these formats, DNA biochips are prepared by depositing DNA fragments with sizes ranging from about a few tens of bases to a few kilobases onto a suitable surface (see, e.g., DeRisi et al., 1996, Nature Genetics 14:457-460; Shalon et al., 5 1996, Genome Res. 6:689-645; Schena et al., 1995, Proc. Natl. Acad. Sci. U.S.A. 93:1053911286; and Duggan et al., Nature Genetics Supplement 21:10-14). For example, in blotting assays, such as dot or membrane DNA/DNA hybridization (Southern Blotting), nucleic acid molecules may be first separated, e.g., according to size by gel electrophoresis, transferred and immobilized to a membrane filter such as a nitrocellulose or nylon membrane, and allowed to hybridize to a single labeled sequence (see, e.g., Nicoloso, M. et al., 1989, Biochemical and Biophysical Research Communications 159:1233-1241; Vernier, P. et al., 1996, Analytical Biochemistry 235:1119). cDNA biochips are prepared by depositing polymerase chain reaction (“PCR”) products of cDNA fragments with sizes ranging from about 0.6 to 2.4 kb, from full length cDNAs, ESTs (expressed sequence tag), etc., onto a suitable surface (see, e.g., DeRisi et al., 1996, Nature Genetics 15 14:457-460; Shalon et al., 1996, Genome Res. 6:689-645; Schena et al., 1995, Proc. Natl. Acad. Sci. U.S.A. 93:10539-11286; and Duggan et al., Nature Genetics Supplement 21:1014). The other biochips format, which is called high-density oligonucleotide biochips, contains thousands of oligonucleotides complementary to defined sequences at defined locations on a surface. These oligonucleotides are synthesized in situ on the surface by, for example, photolithographic techniques (see, e.g., 20 Fodor et al., 1991, Science 251:767-773; Pease et al., 1994, Proc. Natl. Acad. Sci. U.S.A. 91:5022-5026; Lockhart et al., 1996, Nature Biotechnology 14:1675; U.S. Pat. Nos. 5,578,832; 5,556,752; 5,510,270; 5,445,934; 5,744,305; and 6,040,138). Methods for generating biochips using inkjet technology for in situ oligonucleotide synthesis are also known in the art (see, e.g., Blanchard, International Patent Publication WO 98/4153 1, 25 published Sep. 24, 1998; Blanchard et al., 1996, Biosensors and Bioelectronics 11:687-690; Blanchard, 1998, in Synthetic DNA Biochips in Genetic Engineering, Vol. 20, J. K. Setlow, Ed., Plenum Press, New York at pages 111-123).

Despite these technological advances, there still exists a need in the art for improved biochips and methods useful for high-throughput gene and gene product characterization (e.g. single nucleotide polymorphism (“SNP”) detection, insertions and/or deletions of a number of continuous single or multiple nucleotides, gene sequencing, gene expression analysis, alternative splice detection, loss of heterozygosity analysis and the detection of any nucleotide containing organism or any remnant thereof). Some of the problems of existing biochips include the size and positioning of probes, the lack of a single assay condition for all probes, the need for multiple probes due to the G/C content leading to dead space on a biochip, the requirement of multiple PCR oligonucleotides for each sequence to be detected, and the need to label each target. The present invention addresses these needs.

### SUMMARY OF THE INVENTION

The present invention is directed to nucleic acid probes capable of forming unlabeled hairpin, biochips containing multiple probes of the invention, and methods of their use. Each polynucleotide probe capable of forming hairpin is an unlabeled single-stranded nucleic acid comprising a target-specific sequence flanked on each ends by two sequences complementary to one another. Each probe is capable of assuming two conformations. One conformation is a “closed” or “hairpin” conformation wherein the complementary probe ends are hybridized to each other to form a “stem” while the target specific sequence forms a “loop” (the target-specific sequence does not hybridize with any other part of the probe). This closed conformation excludes the binding of other complementary sequences to the arms (the ends of the probe, forming the stem). The second conformation is an “open” conformation wherein the target-specific portion forming the loop hybridizes with the target molecule and causes the two complementary ends to disassociate from one another. This open conformation excludes the re-annealing of the two complementary ends and allows other sequences complementary to the ends to hybridize with said ends. In a preferred embodiment, target hybridization is detected with the use of a “reporter” molecule, which is specifically designed to hybridize with at least one of the free probe ends of the invention.

Each hairpin probe of the invention may be designed to discriminate a specific target molecule that has a single nucleotide variation (e.g., substitution, deletion or insertion of a single nucleotide) or variations of several nucleotides separated by short sequences as well as large insertions and deletions. Because each hairpin probe is designed to open when it hybridizes with the target it was specifically designed for, allowing to distinguish and detect targets. The discrimination capability allows multiple sequences to be screened in a single solution. In a preferred embodiment, a biochip comprises two or more probes of the invention; all perfect hybrids formed upon association of target-specific sequences with the target molecules have a melting temperature equal within a range of 4° C. In another preferred embodiment, all perfect hybrids formed upon association of target-specific sequences with the target molecules have a melting temperature equal within a range of 3° C. More preferably, all perfect hybrids formed upon association of target-specific sequences with the target molecules have a melting temperature equal within a range of 1° C.

In an other preferred embodiment, the target-specific sequence for each probe according to the invention is between 6 and 30 nucleotides in length, preferably between 10 and 25 nucleotides in length, more preferably between 15 and 20 nucleotides in length. In a preferred embodiment, each of the ends comprising the stem of each hairpin probe according to the invention consists of a sequence of at least 10 nucleotides in length, preferably between 5 and 9 nucleotides in length.

Therefore, the invention concerns a nucleic acid probe capable of forming unlabeled hairpin comprising:

(a) a target-specific sequence that is from 6 to 30 nucleotides in length;

(b) a first arm that is less than 10 nucleotides in length and is 5′ of the target specific sequence; and

(c) a second arm that is less than 10 nucleotides in length and is 3′ of the target specific sequence,

wherein the target-specific sequence is not complementary with any other portion of the said probe; further wherein the first arm and the second arm are perfectly complementary to each other.

In a preferred embodiment, the target-specific sequence is from 10 to 25 nucleotides in length, and more preferably, 15 to 20 nucleotides. Preferably, when the target-specific sequence hybridizes with a target molecule, the probe takes an “open” conformation and a “reporter” molecule may hybridize with the first or second arm. “Reporter” molecule comprises less than 10 nucleotides which are perfectly complementary with the nucleic acid sequence of the first arm or the second arm. The “reporter” molecule comprises a detectable marker. The detectable marker may be a nucleotide analogue, a fluorescent label, biotin, imminobiotin, an antigen, a cofactor, dinitrophenol, lipoic acid, an olefinic compound, a polypeptide, an electron-rich molecule, an enzyme, or a radioactive isotope. Preferably, the Dtm (the difference between melting temperature (Tm) of perfect hybrid formed upon association of the target-specific sequence with the target sequence and melting temperature (Tm) of perfect hybrid formed by association of the first and second arm) is greater than 10, more particularly is 15. Alternatively, the Dtm is lower than 10.

The invention further concerns a composition comprising at least one probe of the invention, and at least one ““reporter”” molecule.

The invention also concerns a biochip of probes capable of forming unlabeled hairpins comprising:

(a) a substrate; and

(b) at least two probes of the present invention.

In a preferred embodiment, the probe is attached to substrate. Alternately, said probe further comprise a linker, and is attached to substrate by means of said linker. The substrate consists of a functionalized glass surface, a functionalized plastic surface, a functional metal surface, a conductive metal surface, a conductive plastic surface, a porous substrate, a porous metal, an optical fiber, a glass fiber derived substrate, silicon dioxide, a functional lipidic membrane, a liposome, or a filtration membrane. More particularly, the functionalized plastic surface may consist in polystyrene; the functionalized metal may be platinum, gold, or nickel; the conductive plastic surface may be a carbon based substrate, this substrate optionally being a polymer; and the porous substrate may be glass. In a particular embodiment, all of the first arms of each probe have an identical sequence. So, one identical “reporter” molecule hybridises with each probe. In a preferred embodiment, all perfect hybrids formed upon association of target-specific sequences with the target molecules have a melting temperature equal within a range of 4° C., and more preferably within a range of 1° C. Preferably, the difference between melting temperature of hybrid formed upon association of the target-specific sequence with the target sequence, and melting temperature of hybrid formed upon association of the target-specific sequence with a molecule for which the target specific sequence is not designed is greater or equal to 5° C., more preferably 8° C. In a preferred embodiment, Dtm of at least two probes are equal within a range of 1° C.

A particular embodiment of the present invention concerns a universal addressing system wherein a standardized biochip of immobilized hairpin probes of the present invention is used together with non-immobilized target-specific hairpin or linear probes for detecting target molecules. Preferably, non-immobilized probes are of hairpin type.

The present invention also concerns an unlabeled universal addressing system comprising:

(a) at least two unlabeled first probes comprising:
(i) a target-specific sequence that is 6 to 30 nucleotides in length; and
(ii) a sequence called tag sequence that is from 10 to 50 nucleotides in length connected to the 5′ end of said target specific sequence,
   said target-specific sequence is not complementary with any other portion of the said unlabeled probe;

(b) a biochip of the present invention.

Preferably, said first unlabeled probes comprise:

(i) a target-specific sequence that is 6 to 30 nucleotides in length;

(ii) a first arm that is less than 10 nucleotides in length and is 5′ of said target-specific sequence;

(iii) a second arm that is less than 10 nucleotides in length and is 3′ of said target-specific sequence; and

(iv) a so-called tag sequence that is from 10 to 50 nucleotides in length, connected to the first arm or the second arm.

wherein the target-specific sequence is not complementary with any other portion of said unlabeled probe; further wherein the first arm and the second arm are perfectly complementary to each other.

Preferably, all the first arms of the first probes present an identic sequence. Then, the same “reporter” molecule can hybridize with one or the other of the first and second probes.

Preferably, the biochip comprises at least two second probes capable of forming unlabeled hairpin, comprising:

(i) a sequence specific to a second target that is 6 to 30 nucleotides in length;

(ii) a second first arm of said second probe that is less than 10 nucleotides in length and is 5′ of said target-specific sequence;

(iii) a second second arm of said second probe that is less than 10 nucleotides in length and is 3′ of said target-specific sequence; and

(iv) a linker connecting the first or second arm to the substrate.

said sequence specific to said target of the second probe is not complementary with any other portion of the said second probe; said first arm and said second arm of said second probe are perfectly complementary to each other; and further wherein said tag sequence of one of the first probes completed with arm sequence connected to this tag is complementary with said second target sequence of one of the second probes.

The present invention also concerns to methods for making probes and biochips of the invention.

The present invention further concerns methods of using probes and biochip of the invention. Specific embodiments of the invention are directed to mutation analysis, sequencing, gene expression analysis, loss of heterozygosity and allelic imbalance analysis and detection of any nucleic acid containing organism or any remnant thereof.

The present invention concerns a method of nucleic acid detection comprising:
(a) contacting ex vivo a nucleic acid sample with a biochip comprising at least two probes of the invention; and
(b) detecting a signal from at least one said probe of the biochip which has adopted an open conformation following contacting in step (a).

The present invention further concerns a method of detecting a genetic variant ex vivo in a nucleic acid sample comprising:

(a) contacting a nucleic acid sample with a biochip comprising at least two probes of the invention, wherein at least one probe of the biochip is a probe specific of a genetic variant, possessing a loop region perfectly complementary with said genetic variant, and

(b) detecting a signal from the probe specific of said genetic variant, the signal detected in step (b) indicating the presence of said genetic variant in nucleic acid sample.

Preferably, the detected genetic variant is a single nucleotide polymorphism.

The present invention concerns a method of detecting ex vivo any nucleic acid containing organism or a remnant thereof comprising:

(a) contacting the nucleic acid sample with a biochip comprising at least two probes of the invention, wherein at least one probe is specific for a nucleic acid of an organism or a remnant thereof; and

(b) detecting a signal from said probe specific of the nucleic acid containing organism,

the signal detected in step (b) indicating the presence of the nucleic acid containing organism or a remnant thereof.

Preferably, the nucleic acid containing organism is a virus or a bacterium.

The present invention further concerns a method of detecting ex vivo an alternative splice product of a gene in a nucleic acid sample comprising:

(a) contacting the nucleic acid sample with a biochip, comprising at least two probes of the invention, wherein at least one probe of the biochip is a probe specific for an exon of the gene or specific for a junction of two exons; and

(b) detecting a signal from the probe specific for an exon of the gene or specific for a junction of two exons,

the signal detected in step (b) indicating the presence of the alternative splice product of the gene in the nucleic acid sample.

Preferably, the nucleic acid sample comprises mRNA, or cDNA.

The present invention also concerns a method of ex vivo sequencing an oligonucleotide comprising:

(a) contacting the sample containing the oligonucleotide with a biochip comprising at least two probes of the invention; and

(b) detecting a signal from at least one probe of the biochip;

the signal detected in step (b) being used in determining the sequence of the oligonucleotide.

Preferably, the signal detected in step (b) of previous described methods is obtained using one “reporter” molecule labeled with a detectable marker. The sample in step (a) may be already labeled with a detectable marker.

The present invention also concerns a method of detecting allelic imbalances and loss of heterozygosity ex vivo in a nucleic acid sample comprising:

(a) amplifying of at least one chromosomal DNA region of microsatellite type, using a pair of primers from at least two nucleic acid samples from biological fluids or tissues, wherein at least one of the samples from cells or tissues has no allelic imbalance or loss of heterozygosity, further wherein each tissue or fluid is differentially labeled during amplification;

(b) eliminating of said primers after amplification;

(c) contacting of said amplification products with a biochip comprising at least two probes of the invention, wherein at least one probe of the biochip is complementary to a primer used for amplifying said chromosomal DNA region; and

(d) detecting the signals from at least one probe of said biochip,

the signal detected in step (d) being used for determining the presence of an allelic imbalance or loss of heterozygosity in one of the nucleic acid samples.

The present invention is directed to kits comprising biochips of probes of the invention for determining the presence or absence of various molecules in biological and medical samples. Each kit contains at least one biochip of probes of the invention and one or several additional reagents required for detecting specific target molecules. In addition, the kit may comprise a set of non-immobilized probes of the invention. Optionnally, the kit can further comprise a “reporter” molecule.

### BRIEF DESCRIPTION OF FIGURES

FIG. 1 depicts hairpin probe embodiments.

FIG. 2 depicts the different conformations of an immobilized hairpin probe.

FIG. 3 depicts how a signal can be generated when a hairpin probe hybridizes with its target sequence.

FIG. 4 depicts detection of a target with a hairpin probe and a polymerase labelling system.

FIG. 5 depicts a universal addressing system.

FIG. 6 depicts the sequences of a mRNA, a variant of this mRNA, and the regions of this mRNA that are targeted for hairpin probes.

FIG. 7 shows nucleic acid sequences used to demonstrate how hairpin probes interact with targets and “reporter” molecules.

FIG. 8 is a bar graph of fluorescent intensities after hybridization of labeled target sequences with hairpin probes.

FIG. 9 is a bar graph of fluorescent intensities after successive hybridization of labeled target sequence with hairpin probes, and then with a “reporter” molecule.

FIG. 10 is a bar graph of fluorescent intensities after successive hybridization of a labeled target sequence with hairpin probes, and then with a “reporter” molecule, followed by the removal of non specific binding.

FIG. 11 is a bar graph of fluorescent intensities after hybridization of several labeled targets with hairpin probes.

FIG. 12 is a bar graph of fluorescent intensities after hybridization of several labeled targets with hairpin probes under a more stringent hybridization condition than that in FIG. 11.

FIG. 13 shows a method of analysis of loss of heterozygosity and allelic imbalance, from sample PCR amplifications steps to hybridisation of PCR products on the biochip.

FIG. 14 depicts discrimination ratio (Rd) of hairpin probes as a function of Tm difference between the stem and the loop (Dtm).

FIG. 15 is a bar graph of fluorescent intensities after hybridization of PCR products from amplification of exon 3 of PMP22 gene, on a hairpin probe biochip.

### DETAILED DESCRIPTION OF THE INVENTION

The present invention concerns probes capable of forming unlabeled hairpin, biochips containing a plurality of probes of the invention, and methods of preparation and use of probes and biochip of the invention. It is described in detail and exemplified below.

Probes Capable of Forming Hairpins

The present invention concerns a biochip of probes capable of forming hairpin comprising:
(a) a substrate; and
(b) at least two unlabeled probes capable of forming hairpins, wherein each unlabeled probe comprises:
   (i) a target-specific sequence that is from 6 to 30 nucleotides in length;
   (ii) a first arm that is less than 10 nucleotides in length and is 5′ of the target specific sequence; and
   (iii) a second arm that is less than 10 nucleotides in length and is 3′ of the target specific sequence
   (iv) a linker connecting the first or second arm to said substrate;
      wherein the target-specific sequence is not complementary with any other portion of the said unlabeled probe; further wherein the first arm and the second arm are perfectly complementary to each other, for each probe.

By “unlabeled” is understood that the probe does not comprise detectable marker. E.g. the probe does not comprise a nucleotide analog, a fluorescent label, biotin, imminobiotin, an antigen, a cofactor, dinitrophenol, lipoic acid, an olefinic compound, a polypeptide, an electron-rich molecule, an enzyme, or a radioactive isotope allowing the direct detection of the probe.

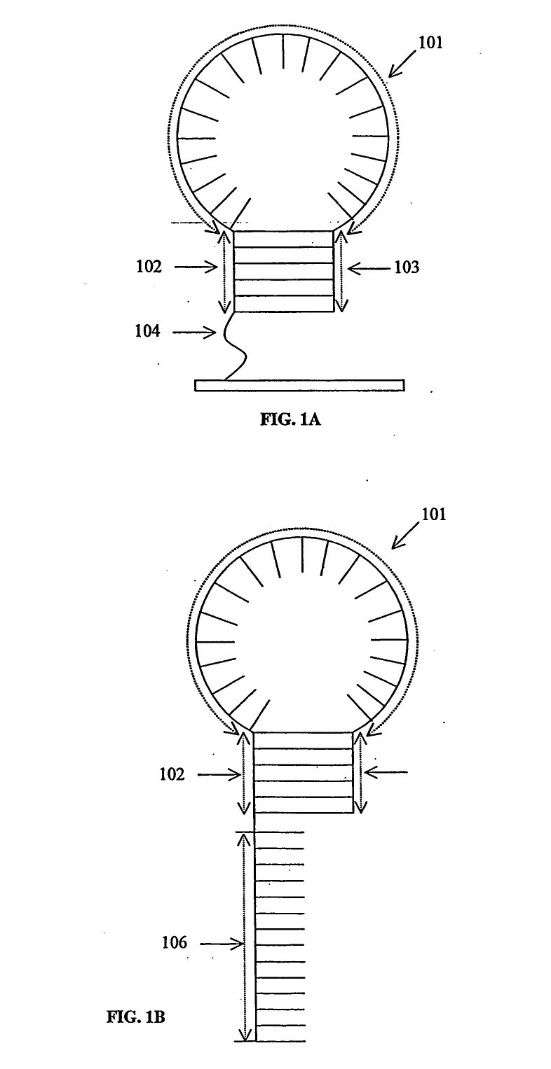

Two examples of unlabeled hairpin probes of the invention are illustrated in FIGS. 1A and 1B. For the purpose of this invention, a hairpin probe is a single-strand nucleic acid comprising a “loop” 101 having a target-specific sequence length of 6 to 30 nucleotides, with a preferred length of 10 to 25 nucleotides and a highly preferred length of 15 to 20 nucleotides, and two ends, or “arms,” which flank the target specific sequence. One arm 102 is attached to the 3′ end of the target-specific sequence and the other arm 103 is attached to the 5′ end of the target-specific sequence. Preferably, each arm comprises less than 10 nucleotides that are perfectly complementary to those of the other arm. More preferably, each arm comprises 4 to 9 nucleotides that are perfectly complementary to those of the other arm. Most preferably, each arm comprises 6-8 nucleotides that are perfectly complementary to those of the other arm. If two or more hairpin probes are to be used together (e.g., in a biochip), all hairpin probes may be designed to have the same arm sequence composition.

The hairpin probes of the present invention may also comprise a “linker” 104. As used herein, a linker refers to a molecule that is connected to one arm of the hairpin probe and is a means for permanently immobilizing the hairpin probe to a substrate 105 such as, but not limited to, a glass slide, for forming a biochip of hairpin probes. As shown in FIG. 1B, the hairpin probes of the present invention may also have a “tag” sequence 106 which is preferably an additional 5-50 nucleotides, more preferably 5-20 nucleotides, and which can be used in a universal address system. Such a sequence tag may be used to “address” a hairpin probe to a specific location on a biochip, by means of its hybridization to an immobilized biochip sequence that is perfectly complementary to the tag or a portion of the tag.

Hairpin Probe Design

Hairpin probes may be designed to be used with a fixed concentration of salts, a fixed temperature, and a fixed concentration of one or more additional chemicals, e.g., formamide, dimethylsulfoxide, tetramethylammonium chloride, and detergents. The fixed temperature for using a hairpin probe may be obtained by first calculating the melting temperature (“Tm”) of the target-specific sequence and target molecule using the “nearest neighbor” Tm calculation method with Meltcalc software (See Schutz, E. et al. 1999. Biotechniques 27:1218-1224.; Allawi H. T. et al. 1998. Biochemistry 37(8):2170-9; Allawi H. T. et al. 1997. Biochemistry 36:10581-94; Peyret N. et al. 1999. Biochemistry 38:3468-77) or (Oligo 6, Molecular Biology Insights, Inc., Cascade, Colo.; Meltcalc, www.meltcalc.de). Meltcalc, (www.meltcalc.de). Meltcalc is spreadsheet software for calculating the thermodynamic melting point of oligonucleotides hybridization with and without mismatches. Meltcalc gives the melting temperature of an oligonucleotide based on varying salts and oligonucleotides concentrations. The preferred Tm of hybrid formed between the target and the target-specific sequence (“Tm of hybrid formed between the target-specific sequence and the target molecule”) is 60+/−10° C. for target-specific sequence concentrations of 100 nM and salts concentration of 100 mM, with sodium (NaCl) and magnesium (MgCl) being the preferred salts.

When two or more hairpin probes are designed to be used together, it is preferred that each Tm of hybrid formed upon association of target-specific sequence with the target molecule of the hairpin probe are more or less equal to those of perfect hybrids formed upon association of target-specific sequences with the target molecules of all other hairpin probes. Preferably, the difference of Tm of hybrids formed upon association of target-specific sequence with the target molecule is equal or lower than 4° C. More preferably, the difference of Tm of hybrids formed upon association of target-specific sequence with the target molecule is equal or lower than 3° C. Even more preferably, the difference of Tm of hybrids formed upon association of target-specific sequence with the target molecule is equal or lower than 2° C. Most preferably, the difference of Tm of hybrids formed upon association of target-specific sequence with the target molecule is equal or lower than 1° C. Variations in each Tm of hybrids formed upon association of target-specific sequence with the target molecule may be obtained by several manipulations of the target-specific sequence. E.g. by, but not limited to, the Tm of hybrids formed between target-specific sequence with the target molecule may be increased or reduced by varying the number of bases in the target-specific sequence. Increasing the number of nucleotides of the target-specific sequence, by increasing the number of nucleotides associated with target molecule, increases the Tm of hybrids formed upon association of target-specific sequence with the target molecule. At the opposite, decreasing the number of nucleotides of the target-specific sequence, by reducing the number of nucleotides associated with target molecule, decreases the Tm of hybrids formed upon association of target-specific sequence with the target molecule. Other means to modify Tm of hybrids formed upon association of target-specific sequence with the target molecule are described thereafter.

Once a loop sequence has been designed, with the Tm of the hybrid between target and the target specific sequence within an acceptable range, the arms may be designed to be complementary to each other and added to the loop sequence. Preferably, the G/C content of the stem is 80% with a preferred value of Tm of 40° C.-80° C. Once each probe has been designed, mfold (Zuker, M. mfold-2.3. ftp://snark.wustl.edu) is used to verify that no additional internal loop is present in the hairpin probe and that the probe is assuming a hairpin conformation only by the association of nucleotides of the stem. Lack of homology between each loop and any other nucleotide sequence to be used in the assay (e.g. a PCR product different from one analysed, a cDNA different from one analysed), is then checked. An alignment of all hairpin probes with each PCR products is performed using alignment tools such as LALIGN (http://fasta.bioch.virginia.edu/fasta/lalign.html), or LFASTA (http://www.2.igh.cnrs.fr/fasta/lfasta-query.html).

For each probe complementary to a sequence to be analysed, the difference between loop Tm and stem Tm is calculated using the following formula:

Dtm=Tm s−Tm I, with Tm s=stem Tm and Tm I=loop Tm, with Tm determined as previously described. More specifically, Tm of the stem is the melting temperature (Tm) of hybrid formed upon association of the first and the second arm of the probe. The Tm of the loop is the melting temperature (Tm) of the perfect hybrid formed between the target molecule and the target specific sequence.

In a preferred embodiment, the Dtm is greater than 10 for each probe, with a preferred value close to 15 (e.g. between 12-17, preferably between 13 and 16 or between 14 and 16); e.g. equal to 15. However, for purposes in which signal sensitivity must be favoured against probe discrimination power, this Dtm may be lower than 10.

For a set of probes used together in an experiment (more specifically as a probe biochip), the distribution of Dtm value for the set of probes must be centred on a value greater than 10, preferably a mean value of 15, with a standard deviation of 5. However, for purposes in which signal sensitivity must be favoured against probe discrimination power, this mean Dtm value may be less than 10, with a standard deviation of 5.

For two probes used together to analyse two alleles of a sequence (polymorphism or mutation), Dtm values of these probes must be as close as possible. Dtm of two probes used for analysing two different alleles of a sequence are equal, within a range of 2, preferably within a range of 1. These Dtm values are preferred values, that are acceptable within a range <1 or >2 for a couple of probe in some instance, when the base composition is not suitable to obtain equal Dtm values.

Hairpin Probe Immobilization and Biochips

Hairpin probes may be immobilized or included on a substrate to form such products as, but not limited to, biochips of hairpin probes that have sequence specificity to one or more target molecules. Hairpin probes may also be immobilized on a variety of substrates such as, but not limited to, beads or sub-micron particles. Each target-specific hairpin probe may be identified after immobilization by a physical, chemical or optical property that differentiates each hairpin probe from the others. The probes may also be directly synthesized on a solid surface, using a two dimensional system or the individual addressing system. For immobilization of hairpin probes to occur, hairpin probes contain at least one attached moiety, a “linker,” as described previously. Hairpin probes having a linker may also be immobilized with a moiety that has affinity for the linker and is attached to a substrate.

Detection of target molecules is preferably measured using biochips of probes of the invention. A biochip is an organization of positionally-addressable binding sites on a support, e.g., a solid substrate, where each hairpin probe is immobilized on the surface of the support. Preferably, substrates used in this invention are, but are not limited to, glass slides modified by silanization in order to create chemical groups suitable for covalent immobilization of hairpin probes. These groups may be, but are not limited to, primary amines, thiols, carboxyls or aldehydes. Other surfaces useful for immobilization may be, but are not limited to, silicon dioxide, plastic polymers such as polystyrene or conductive supports like metallic surfaces or glassy carbon. The support may be porous or non-porous. For example, hairpin probes may be attached to a nitrocellulose or nylon membrane or filter. Specific embodiments of the present invention are directed to unlabelled hairpin probes wherein the substrate consists of a functionalized glass surface obtained by silanization with a silane bearing a moiety capable of reacting with aminated or thiolated probes, to create a covalent link, a functionalized plastic surface obtained by creating functional groups suitable for reacting with aminated or thiolated probes, to create a covalent link, like chemical or electrochemical oxidation, a conductive metal surface, a conductive plastic surface, a porous substrate (glass preferred), a porous metal, an optical fiber, a glass fiber derived substrate, silicon dioxide, a functional lipidic membrane, a liposome, or a filtration membrane. More particularly, the preferred functionalized plastic surface is polystyrene; the preferred functionalized metal is platinum, gold, or nickel; the preferred conductive plastic surface is a carbon based substrate, and this preferred carbon substrate is a polymer.

Such methods for attachment of probes are well known in the art (see, e.g., Cass et al, eds., 1998, Immobilized Biomolecules in Analaysis, A practical approach, Ed., Oxford University Press, Great Clarendon Street, Oxford).

Hairpin probes Biochips may be made using any method known in the art. However, produced biochips generally share certain characteristics. The hairpin probes biochips of the invention are reproducible, allowing multiple copies of a given biochip to be produced and easily compared with each other. Immobilization of hairpin probes on a biochip can be obtained through fluid transfer with a conventional arrayer, or electrokinetic transfer or electropolymerisation of electroactive oligonucleotides, by all means of direct chemical bonding of probes on a surface, of inclusion of the probe into a solid support, or in situ synthesis via photolithography using a pre-manufactured set of masks (using modified bases adapted to this process). Biochips of the invention are made from materials that are stable under the conditions used for nucleic acid hybridization. The biochips are preferably small, e.g., between 1 cm² and 25 cm², preferably 1 to 3 cm². However, both larger and smaller biochips are also contemplated in this invention. Large biochips may be preferable for simultaneously evaluating a very large number of different targets. The density of hairpin probes on a biochip of the present invention may vary. The density may range from several (e.g. 3, 10, 30) up to 100 different (i.e., non-identical) probes per 1 cm² or higher.

In the present invention, hairpin probes may be immobilized on a solid surface as a biochip of probes that possess different target sequence specificity. The immobilization of the probes is performed such that the probes do not inhibit the ability of each probe's arms to associate with itself or with target molecules or other substances used in an assay. As noted above, the probes of the present invention are designed to assume two types of conformational structure depending of the assay conditions and the presence or absence of complementary nucleic acid sequences. One structure is the “closed” conformation (hairpin) for which both arm sequences are hybridized to one another. This structure is obtained in favourable assay conditions and in absence of a target molecule that associates with the hybridization probe's target-specific sequence. The second structure is the “open” conformation where arm sequences are not hybridized. This conformation may be detected when the target-specific sequence associates with its specific target. A preferred method for forming a hairpin biochip is by attaching the probes of the invention to a surface by direct contact or “printing” on glass plates, as is described generally by Schena et al., 1995, Science 270:467-470. This method is especially useful for preparing biochips of cDNA (see, DeRisi et al., 1996, Nature Genetics 14:457-460; Shalon et al., 1996, Genome Res. 6:639-645; and Schena et al., 1995, Proc. Natl. Acad. Sci. U.S.A. 93:10539-11286). Other methods for making biochips, e.g., by masking (Maskos and Southern, 1992, Nucl. Acids. Res. 20:1679-1684), may also be used. In principle, and as noted supra, any type of biochip, for example, “dot blots” on a nylon hybridization membrane (see Sambrook, J. et al., eds., 1989, Molecular Cloning: A Laboratory Manual, 2nd Ed., Cold Spring Harbor Laboratory Press, Cold Spring Harbor, N.Y.) may be used.

Biochips of the present invention may be manufactured by means of an ink jet printing device for oligonucleotide synthesis, e.g., using the methods and systems described by Blanchard in International Patent Publication No. WO 98/41531, published Sep. 24, 1998; Blanchard et al., 1996, Biosensors and Bioelectronics 11:687-690; Blanchard, 1998, in Synthetic DNA Biochips in Genetic Engineering, Vol. 20, J. K. Setlow, ed., Plenum Press, New York at pages 111-123; and U.S. Pat. No. 6,028,189. Specifically, the hairpin probes in such biochips are preferably synthesized, e.g., on a glass slide, by serially depositing individual nucleotide bases in “microdroplets” of a high surface tension solvent such as propylene carbonate. The microdroplets have small volumes (e.g., 100 pL or less, more preferably 50 pL or less) and are separated from each other on the biochip (e.g., by hydrophobic domains) to form circular surface tension wells which define the locations of the biochip elements (i.e., the different probes). Polynucleotide hairpin probes may be attached to the surface covalently at the 3′ end or 5′ end of the hairpin polynucleotide.

Target Molecules

Target molecules which may be analyzed by the methods and compositions of the invention include nucleic acid molecules, particularly RNA molecules such as, but not limited to, messenger RNA (mRNA) molecules, ribosomal RNA (rRNA) molecules, cRNA molecules (i.e., RNA molecules prepared from cDNA molecules that are transcribed in vitro) and fragments thereof. Target molecules which may also be analyzed by the methods and compositions of the present invention include, but are not limited to DNA molecules such as genomic DNA molecules, cDNA molecules, and fragments thereof including oligonucleotides, ESTs, STSs, microsatellite sequences, etc. Other target molecules which may also be analyzed by the methods and compositions of the present invention include, but are not limited to proteins, complexes including proteins, other molecules, and complexes of such other molecules having affinity for DNA, e.g., transcription factors, proteins of the DNA repair system, and anti-DNA antibodies (Fang, W. et al. 2000. Anal. Chem. 72:3280-5; Bar-Ziv, R. et al. 2001. P.N.A.S. U.S.A. 98:9068-73; Hamaguchi, N. et al. 2001. Anal. Biochem. 294:126-3 1). For transcription factors, hairpin probes could be used to determine which putative affinity sequence for a trancription factor can be designed within the loop of a hairpin probe and used to determine which sequence has the higher affinity for the transcription factor.

The target molecules may be from any source. For example, the target molecule molecules may be naturally occurring nucleic acid molecules such as genomic or extra genomic DNA molecules isolated from an organism, or RNA molecules, such as mRNA molecules, isolated from an organism. Alternatively, the target molecules may be synthesized. These target molecules can be for example nucleic acid molecules synthesized enzymatically in vivo or in vitro, such as cDNA molecules, or polynucleotide molecules synthesized by PCR, RNA molecules synthesized by in vitro transcription, etc. PCR methods are well known in the art, and are described, for example, in Innis et al., eds., 1990, PCR Protocols: A Guide to Methods and Applications, Academic Press Inc., San Diego, Calif. The sample of target molecules may comprise, e.g., molecules of DNA, RNA, or copolymers of DNA and RNA. The target molecules of the present invention may correspond to particular genes, to particular gene transcripts, or to particular fragments of a gene transcript (e.g., to particular mRNA sequences expressed in cells or to particular cDNA sequences derived from such mRNA sequences).

Target molecules to be analyzed may also be prepared in vitro from nucleic acids extracted from cells. For example, RNA may be extracted from cells (e.g., total cellular RNA, poly(A)+ messenger RNA, or fractions thereof) and messenger RNA purified from the total extracted RNA. Methods for preparing total and poly(A)+ RNA are well known in the art, and are described generally, e.g., in Sambrook et al., supra. RNA may be extracted from cells of the various types of interest in this invention using guanidinium thiocyanate lysis followed by CsCl centrifugation and an oligo dT purification (Chirgwin et al., 1979, Biochemistry 18:5294-5299). RNA may also be extracted from cells using guanidinium thiocyanate lysis followed by purification on RNeasy columns (Qiagen). cDNA may then be synthesized from the purified mRNA using, e.g., oligo-dT or random primers. The target molecules may be cRNA prepared from purified messenger RNA or from total RNA extracted from cells. As used herein, cRNA is defined here as RNA complementary to the source RNA. The extracted RNAs may then be amplified using a process in which doubled stranded cDNAs are synthesized from the RNAs using a primer linked to an RNA polymerase promoter in a direction capable of directing transcription of anti-sense RNA. Antisense RNAs or RNAsc may then be transcribed from the second strand of the double stranded DNAsc using an RNA polymerase (see, e.g., U.S. Pat. Nos. 5,891,636; 5,716,785; 5,545,522 and 6,132,997). Oligo-dT primers containing an RNA polymerase promoter may be used. Total RNA may be used as input for cRNA synthesis. An oligo-dT primer containing a T7 RNA polymerase promoter sequence may be used to prime first strand cDNA synthesis, and random hexamer primers may be used to prime second strand cDNA synthesis by MMLV Reverse Transcriptase. This reaction yields double-stranded cDNA that contained the T7 RNA polymerase promoter at the 3′ end. The double-stranded cDNA may then transcribed into cRNA by T7 RNA polymerase. The concentration of a synthesized target may vary in concentration from 1 μM to 50 nM. The concentration of target synthesized by PCR may vary from 5 ng/μL to 50 ng/μL.

The target molecules may be PCR primers, or comprised in PCR primers. Thus, probes of the invention may be used to detect PCR product amplification. More specifically, this detection constitutes an excellent negative blank of PCR. These negative blanks are performed by amplifying sequence without genomic DNA, but in presence of reagents needed for PCR and primer pair, at least one comprising a sequence perfectly complementary to target-specific sequence of the hairpin probe.

The target molecules to be analyzed by the methods and compositions of the invention may be, but need not be, detectably labelled. For example, cDNA may be labelled directly, i.e., with nucleotide analogs, or indirectly, e.g., by making a second, labelled cDNA strand using the first strand as a template. Alternatively, the double-stranded cDNA may be transcribed into cRNA and labelled during its transcription. The detectable label may be a fluorescent label, e.g., by incorporation of nucleotide analogs. Other labels suitable for use in the present invention may include, but are not limited to, biotin, imminobiotin, antigens, cofactors, dinitrophenol, lipoic acid, olefinic compounds, detectable polypeptides, electron rich molecules, enzymes capable of generating a detectable signal by action upon a substrate, and radioactive isotopes. Preferred radioactive isotopes include, but are not limited to, ³²P, ³⁵S, ¹⁴C, ¹⁵N and ¹²⁵I. Fluorescent molecules suitable for use in the present invention include, but are not limited to, fluorescein and its derivatives, rhodamine and its derivatives, texas red, 5′carboxy-fluorescein (“FMA”), 2′,7′-dimethoxy-4′,5′-dichloro-6-carboxy-fluorescein (“JOE”), N,N,N′,N′-tetramethyl-6-carboxy-rhodamine (“TAMRA”), 6′carboxy-X-rhodamine (“ROX”), HEX, TET, IRD40, and IRD41. Additional fluorescent molecules that are suitable for use in the invention further include: cyanin dyes (including by not limited to Cy3, Cy3.5 and Cy5), BODIPY dyes (including but not limited to BODIPY-FL, BODIPY-TM, BODIPY-630/650, and BODIPY-650/670), and ALEXA fluorescent dyes (including but not limited to ALEXA-488, ALEXA-532, ALEXA-546, ALEXA-568, and ALEXA-594), as well as any other fluorescent dye which is known to those skilled in the art. Electron rich indicator molecules suitable for the present invention include, but are not limited to, ferritin, hemocyanin, and/or colloidal gold. Alternatively, the target molecules may be labelled by specifically complexing a first group to the target molecules. A second group, covalently linked to an indicator molecules and which has an affinity for the first group, may be used to indirectly detect the target molecules. In such an embodiment, compounds suitable for use as a first group may include, but are not limited to, biotin and iminobiotin. Compounds suitable for use as a second group include, but are not limited to, avidin and streptavidin. Fluorescent DNA intercalatant species like ethidium bromide or sybr green I dye may also be used to monitor fluorescence increase of the signal upon hybridization of target polynucleotides with a hairpin probe or hybridization of hairpin probes and “reporter” molecule (see below). This embodiment may be particularly adapted to analyze hybridization in real time.

In a preferred embodiment, however, target molecules are unlabelled and the detectable labels described above are attached to a “reporter” molecule (see below).

Hybridization Methods Available for Use with Hairpin Probes

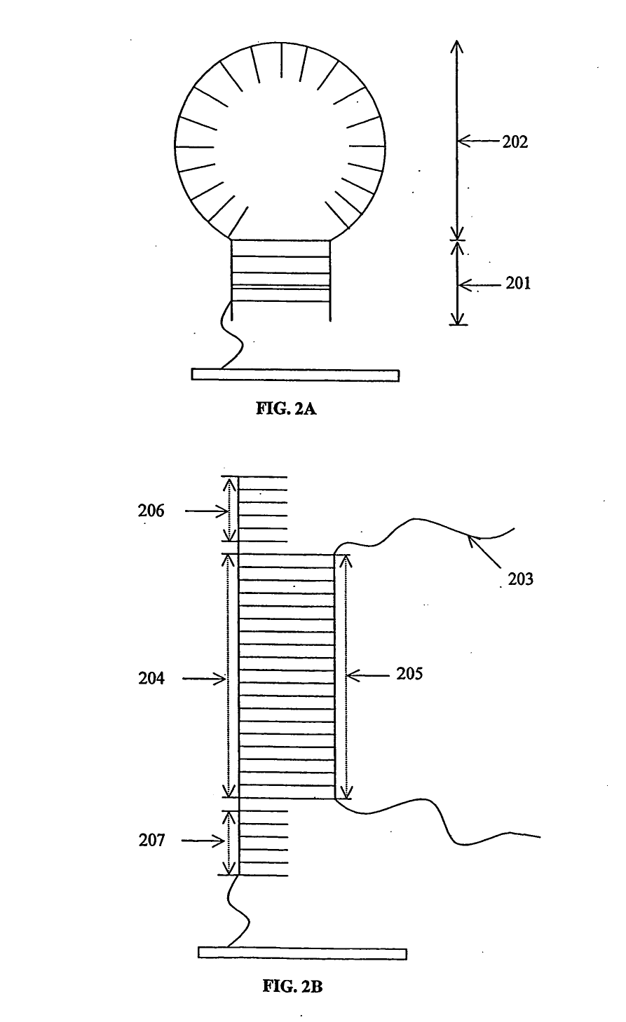

The hairpin probes of the present invention are designed to adopt two alternative conformations when used to screen for target molecules. These two conformations, a closed” conformation and an “open” conformation, are shown in FIG. 2. Both FIG. 2A and FIG. 2B show a hairpin probe linked to a substrate. FIG. 2A shows a hairpin probe in the closed conformation. When the specific target of a hairpin probe is not present, the arms of the probe hybridize to one another to form a “stem” 201 and the target-specific sequence forms a “loop” 202. No other conformation of the hairpin probe is possible because the hairpin probe arms are designed to hybridize only with each other. The preferred fixed temperature for screening with a hairpin probe is 5 to 10° C. below the Tm of hybrid formed upon association of the loop target-specific sequence with its target molecule. This temperature may vary depending on the probe length and presence or absence of additional mutations present in the loop structure. A longer or shorter target specific sequence or stem sequence may be used to modify the optimal hybridization temperature. Upon association with a target molecule, the hairpin probe undergoes a structural change. The association of a portion of the loop sequence with its target molecule (i.e., annealing of a nucleic acid target-specific sequence and its target nucleic acid) forces the hairpin structure to open.

Competition between the hybridization of the complementary arms with each other and the target-specific sequence with its target molecule is key for discriminating between perfect association of the target-specific sequence and its target molecule and imperfect association with other target molecules. When a hairpin probe is exposed to the target molecule it was designed for (i.e., a perfect match), the interaction between the target-specific sequence and the target molecule causes the disassociation of the stem, thereby changing the conformation of the hairpin probe into the “open” position. FIG. 2B shows a hairpin probe in an “open” conformation resulting from association with its target molecule 203. The target-specific sequence 204 hybridizes with a portion 205 of the target molecule 203. It is envisioned that the present invention may encompass an interaction between a hairpin probe and a target sequence where the entire portion of the loop hybridizes with the entire target molecule or a lesser portion of the target molecule. This hybridization between the target-specific sequence 204 and the target molecule 203 does not have to include the entire target molecule 203. When the target-specific sequence 204 is hybridized with a portion 205 of the target molecule 203, the arms of the probe, the arm 206 on the 5′ side of the target-specific sequence and the arm 207 on the 3′ side of the target-specific sequence, disassociate and are accessible to interact with “reporter” molecules.

Binding affinity of target nucleic acids to probe sequences during hybridization depends on both the sequence similarity of different target sequences in a sample and the hybridization stringency conditions, i.e., the hybridization temperature, the salt concentrations, and the presence of chemicals that reduce affinity. Binding kinetics also depends on the relative concentrations of different nucleic acids in a sample. For polynucleotide probes targeting (i.e., complementary to) low-abundance species, or targeting nucleic acid having highly similar (i.e., homologous) sequences, such “cross-hybridization” can significantly contaminate and confuse the results of hybridization measurements. For example, cross-hybridization is a particularly significant concern in the detection of SNP's since the sequence to be detected (i.e., the particular SNP) must be distinguished from other sequences that differ by only a single nucleotide.

Several approaches have been devised in the art to reduce cross-hybridization and may be used in the methods of the invention. Cross-hybridization can be minimized by regulating either the hybridization stringency conditions during hybridization and/or during post-hybridization washes. For example, “highly stringent” wash conditions may be employed to destabilize all but the most stable duplexes such that detected signals represent perfect matches between specific sequence and a hairpin probe target-specific sequence. Exemplary highly stringent conditions include, e.g., hybridization DNA in 5×SSC buffer, 1% sodium dodecyl sulfate (SDS), 1 mM EDTA at 65° C., and washing in 0.1×SSC buffer/0.1% SDS (Ausubel et al., eds., 1989, Current Protocols in Molecular Biology, Vol. I, Green Publishing Associates, Inc., and John Wiley & Sons, Inc., New York, N.Y., at p. 2.10.3). These washes may be performed at 5-10° C. below the Tm of hybrid formed between the target-specific sequence and the target molecule. Highly stringent conditions allow detection of allelic variants of a nucleotide sequence, e.g., about 1 mismatches per 10-30 nucleotides. Alternatively, “moderate-” or “low-stringency” wash conditions may also be used. Moderate- or low-stringency conditions are also well known in the art (see, e.g., Sambrook, J. et al., eds., 1989, Molecular Cloning: A Laboratory Manual, 2nd Ed., Cold Spring Harbor Laboratory Press, Cold Spring Harbor, N.Y., at pp. 9.47-9.51 and 11.55-11.61; Ausube let al., eds., 1989, Current Protocols in Molecular Biology, Vol I, Green Publishing Associates, Inc., John Wiley & Sons, Inc., New York, at pp. 2.10.1-2.10.16). Exemplary moderately stringent wash conditions include, e.g., washing in 0.2×SSC buffer 0.1% sodium dodecyl sulfate (SDS), at 42° C. (Ausubel et al., 1989, supra). Exemplary low-stringency washing conditions include, e.g., washing in 5×SSC buffer or in 0.2×SSC/0.1% SDS at room temperature (Ausubel et al., 1989, supra).

Contributions of cross-hybridization to hairpin signals can be monitored and removed by subtracting signals from suitable reference probes. For example, to measure the contribution of cross-hybridization, hairpin probes that do not hybridize to any of the targets may be used in the assay. This non-specific hybridization can then be subtracted from the specific hybridization signal.

Cross-hybridizations can also be reduced by using probes of the invention having weak differences of Tm of hybrids formed upon association of target-specific sequence with the target sequence, i.e. differences equal or lower than 1° C. At the opposite, when differences of Tm of hybrids formed upon association of target-specific sequence with the target sequence increase, i.e. higher than 1° C., cross-hybridizations become a more significant factor. In such case, similar approaches as previously described may be employed, the number of hairpin probes used to screen each target molecule may be increased, or a combination of these approaches may be used.

One or more unique control sequence may be used and the temperature may be determined by analysing which hairpin probe hybridizes with the control sequence. This control sequence may be complementary to 5-10 different hairpin probes, which differ from each other by the length of the loop sequence and thereby differ in Tm. For example, one hairpin probe may be complementary to eight nucleotides localized in the centre of the control sequence and have a Tm of hybrid formed between target and target-specific sequence hybridization temperature of 40° C. A second hairpin may be complementary to 12 nucleotides of the control sequence and have a Tm of hybrid formed between target and target-specific sequence of 45° C. Additional hairpin probes may be complementary to other regions of the control sequence and have other Tm's of hybrid formed between target and target-specific sequence. It is then possible for a user to determine assay temperature based on identification of the hairpin probe that is hybridised with sequence control.

The invention concerns a biochip of probes of the invention, wherein all perfect hybrids formed upon association of target-specific sequence with the target molecule have a melting temperature equal, more or less 4° C.

The invention is also directed to a biochip of probes of the invention; wherein each hybrids formed upon association of target-specific sequence of each probe with the target molecule have a melting temperature equal within 1° C. to melting temperature of hybrids formed upon association between target-specific sequence and target molecule of all other probes.

The invention is further directed to a biochip of probes of the invention, wherein the difference between each melting temperature of hybrid formed upon association of the target-specific sequence with the target sequence, and a second melting temperature for association of the target-specific sequence with a molecule for which the target specific sequence is not designed is greater or equal to 5° C. The difference between each melting temperature of hybrid formed upon association of the target-specific sequence with the target sequence, and the second melting temperature may be, without any limitation greater or equal to 8° C.

Reporter Molecules

The present invention also encompasses one or more unattached labelled or unlabelled “reporter” molecules such as a nucleic acid sequences comprising a length equal to that of a probe arm. The invention is thus directed to a biochip of probes of the invention wherein a “reporter” molecule may hybridise with any of the hairpin probes. A hairpin probe of the present invention may be used in conjunction with “reporter” molecules, which may only hybridize to the open conformation of the probe, thus differentiating the open and closed conformation and generating a signal only when a target molecule associates with the target-specific sequence of a hairpin probe.

“Reporter” molecules may be designed to have a loop stem structure with a 4-10 nucleotide stem, preferably 6 nucleotides or less, and a loop comprising 12-20 nucleotides where the target-specific sequence of said loop may be fully complementary to one arm of a hairpin probe.

“Reporter” molecules may be designed to have a linear structure comprising a sequence fully complementary of one arm of a hairpin probe.

The invention is thus directed to a biochip of unlabelled hairpin probes, wherein a target molecule is hybridised with target-specific sequence, and further wherein a “reporter” molecule is hybridised with first or second arm. The “reporter” molecule comprises preferably less than 10 nucleotides fully complementary with nucleic acid sequence of first or second arm.

The “reporter” molecules may also be designed with a minor-grove binder attached to one end of the molecule. Labelling of the reporter molecules may be accomplished in a manner similar to that described for labelling of target sequences (see above) with a fluorescent labelling at the 3′ and/or 5′ end.

Once a sample has be exposed to a hairpin probe or a set of hairpin probes, a probe arm may become accessible to a complementary “reporter” molecule as a result of association of a loop with its target sequence and a signal may be generated. The “reporter” molecule may be, but is not limited to, a nucleic acid sequence, a peptide nucleic acid sequence, a locked nucleic acid, an oligopeptide sequence, a protein, or enzyme coupled to a nucleic acid sequence. The reporter molecule is preferably characterized by its ability to bind an arm of a probe of the invention only in the “open” conformation. The reporter molecule preferably contains at least a sequence perfectly complementary to one of the arm sequences of a hairpin probe of the invention, only in the “open” conformation. The “reporter” molecule comprises preferably at least one sequence fully complementary with one of arm sequence of a hairpin probe. Upon addition of a reporter molecule, association between the “reporter” molecule and a hairpin probe occurs only with an arm of the probe that is in the open structure conformation. This open structure is a consequence of the association of the loop of a hairpin probe with its specific target molecule. For hairpin probes immobilized on the same substrate, the hairpin probe may be designed to have the same arm sequence so that only one “reporter” molecule is needed for analysis of the association of all hairpin probes with their specific target molecules.

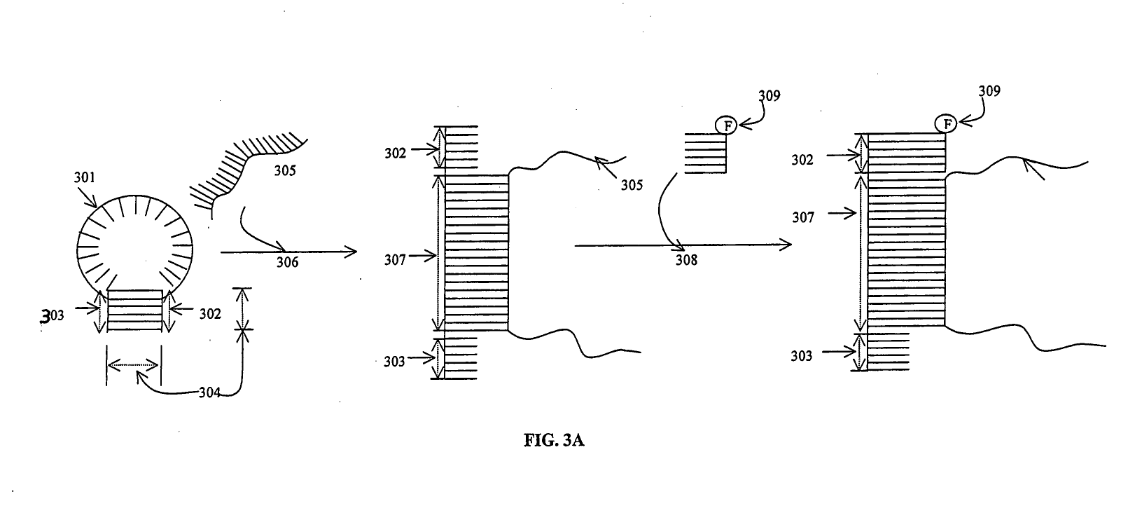

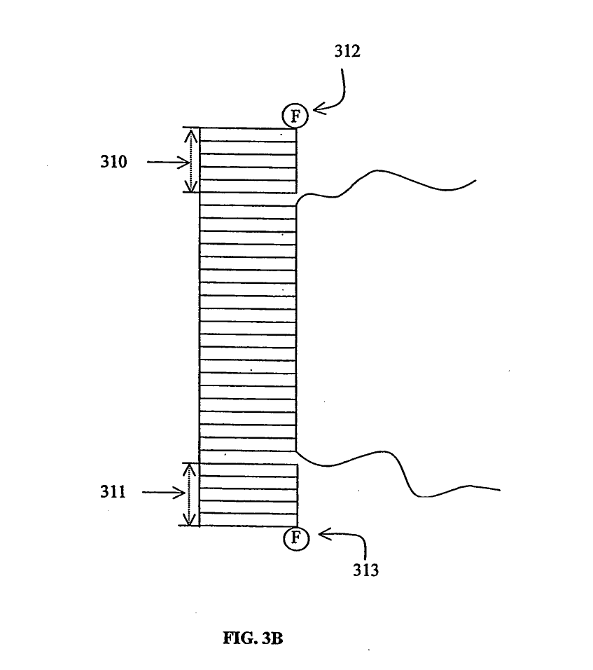

FIG. 3 shows how a “reporter” molecule associates with a hairpin probe of the invention to generate a signal upon hybridization of the hairpin probe with its target molecule. FIG. 3A shows a progression of how a “reporter” molecule associates with a hairpin probe. First, a hairpin probe 301 is in a closed conformation wherein the arm 302 that is 5′ of the target-specific sequence and the arm 303 that is 3′ of the target-specific sequence hybridize to form the stem 304. With the exposure 306 to the target molecule 305 (i.e. a nucleic acid) for which hairpin probe has been designed, the target-specific sequence 307 hybridizes with the target molecule 305. This causes the hairpin probe to assume an open conformation wherein the arms 302 and 303 of the hairpin probe disassociate from each other and are accessible to a “reporter” molecule. With the addition 308 of a “reporter” molecule 309, comprising a nucleic acid, to the probe of the invention in an open conformation, the “reporter” molecule 309 hybridizes with the arm 302 that is 5′ of the target-specific sequence because a portion of the “reporter” molecule 309 was designed to be the complement of the arm 302 that is 5′ of the target-specific sequence.

Because each hairpin probe has two arms, two different “reporter” molecules may be designed and used for generating a signal when a hairpin probe hybridizes with a target sequence. FIG. 3B depicts an open conformation probe of the invention where both arms 310 and 311 of the hairpin probe are hybridized to two “reporter” molecules 312 and 313. When two “reporter” molecules are used and one “reporter” molecule is designed to contain a nucleic acid sequence that is complementary to one arm of the hairpin probe and the second “reporter” molecule designed to contain a nucleic acid sequence that is complementary to the other arm of the hairpin probe, it may be necessary that the “reporter” molecules be added to the hairpin probe consecutively with a washing step between the steps of adding the “reporter” molecules. This is due to the fact that each “reporter” molecule contains a nucleic acid sequence that is perfectly complementary to that of the other “reporter” molecule. If both “reporter” molecules were added to the hairpin probe together, the “reporter” molecules may hybridize to each other as well as to the hairpin probe.

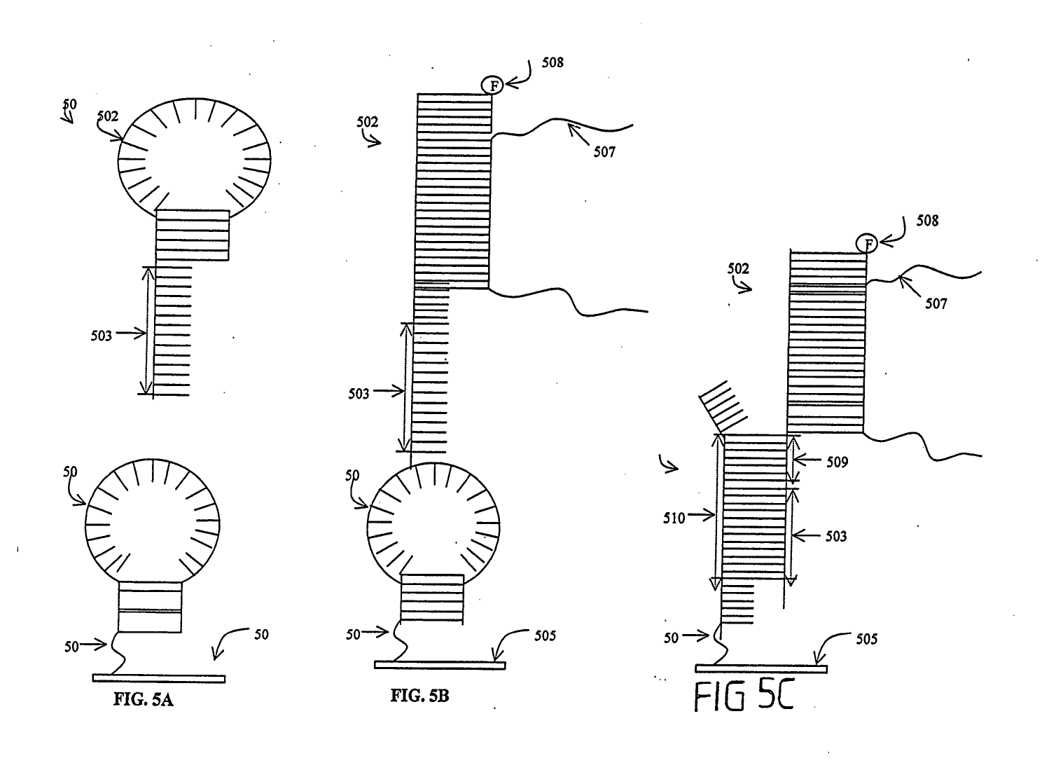

The “reporter” molecules may comprise a detectable marker. Preferably, detectable marker may be a nucleotide analog, a fluorescent label, biotin, imminobiotin, an antigen, a cofactor, dinitrophenol, lipoic acid, an olefinic compound, a polypeptide, an electron-rich molecule, an enzyme, or a radioactive isotope. “Reporter” molecules may be nucleotide sequences labelled, e.g. with a fluorochrome (referred to as F in FIGS. 3A, 3B, 5B, and 5C) or unlabelled sequences having additional modifications resulting in increased affinity for the arms they are to associate with. For example, this modification may be a DNA minor groove binder molecule attached to either 3′ or 5′ of the “reporter”, or a chemical modification of the “reporter” that allows a covalent bonding between DNA hybridized strands upon specific treatment. These modifications may all include the incorporation of a phosphorethioate moiety into the phosphate backbone of “reporter” molecule (Cogoi, S. et al. 2001. Biochemistry 40:1135-43; Xodo, L. et al. 1994 Nucleic Acids Res. 22:3322-30).

The “reporter” molecules may also be detected by their mass, using mass spectrometry and time of flight analysis. For example, probes of the invention immobilised on a substrate and hybridized with target sequences and “reporter” molecules may be directly analyzed by mass spectrometry by orienting a laser source of the mass spectrometer on the discrete region the solid surface corresponding to one specific probe, and collecting the mass and relative abundance of each “reporter” molecule. The “reporter” molecule may vary in length and in mass, or may be attached to several molecular species of known weight, detectable by mass spectrometry (Laken, S. J. et al. 1998. Nat. Biotechnol. 16:1352-6; Little, D. P. et al. 1997. Nat. Med. 3:1413-6; Little, D. P. 1997. Eur. J. Clin. Chem. Clin. Biochem. 35:545-8; Braun, A. et al. 1997. Clin. Chem. 43:1151-8).

The “reporter” molecule may be coupled to electrochemically active molecules like ferrocene and cobaltocene derivatives that generate an oxydo-reduction electric current when a potential is applied onto them (Umek, R. M. et al. 2001. J. Mol. Diagn. 3:74-84; Padeste, C. et al. 2000. Biosens Bioelectron. 15:431-8; Tsai, W. C. et al. 1995. Analyst 120:2249-54). The “reporter” molecules may also be an enzyme capable of generating electro active species, either by electrocatalytic activity or by cleavage of an electrochemically inactive substrate into an electrochemically active product (Valat, C. et al. 2000. Analytica Chimica Acta 404:187-94; Oliver, B. et al., 1997. Anal. Chem. 69:4688-94; Limoges, B. 1996. Anal. Chem. 68:4141-48; Bourdillon, C. et al. 1996. J. Am. Chem. Soc. 115:1226469). For such a “reporter” molecule, probes of the invention may be immobilised on conductive surfaces like silicon dioxide, graphite, glassy carbon, indium-selenium oxide, metallic surfaces or plastic based conductive polymers, or non conductive surfaces presenting discrete areas of pre-deposited or pre-polymerized conductive materials. This detection method may use electropolymerization of detector or part of it on the solid surface as a mean for detecting a signalling current, or a signalling potential.

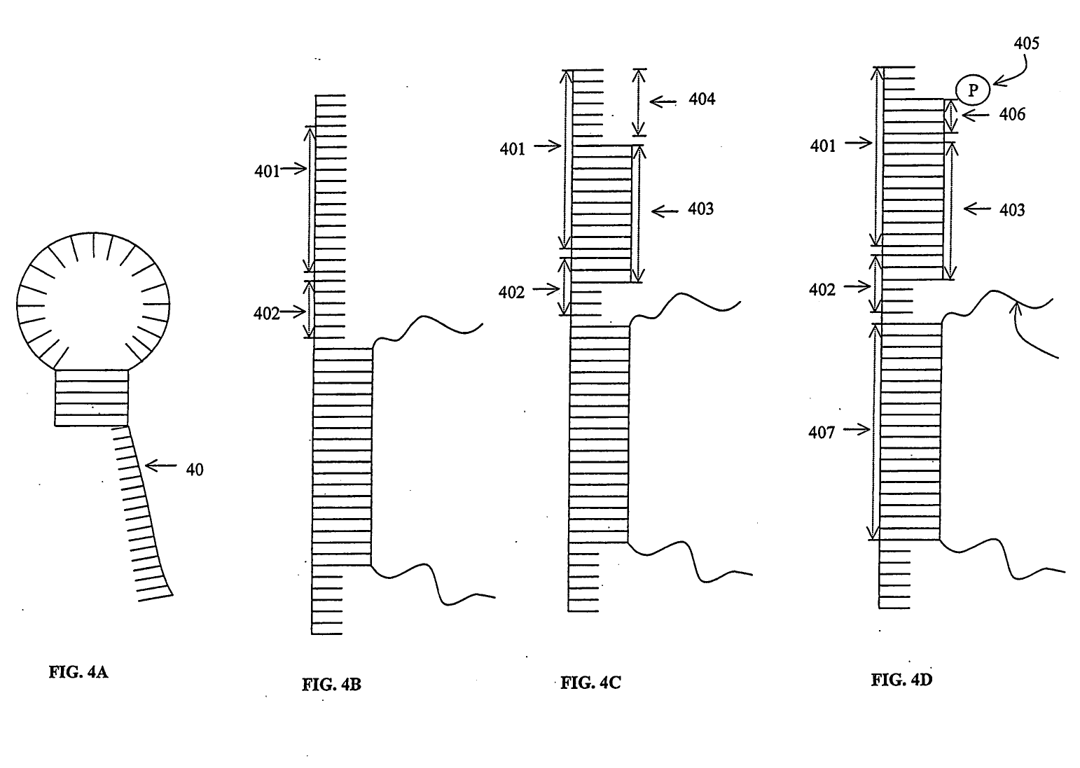

Another “reporter” system by which the association of a hairpin probe with a target molecule can be detected is with a polymerase labelling system. FIG. 4 depicts how such a system may work. FIG. 4A shows a hairpin probe in a closed conformation with an additional 10 to 20 nucleotide sequence, the “primer” 401, on the 5′ end of the probe. Preferably, the primers on all hairpin probes to be used in a specific test condition are designed to have the same nucleotide sequence. The present invention also encompasses the use of different primers in a specific test condition to add another level of differentiation between the signals generated upon hybridization of a hairpin probe with its specific target molecule. For any primer used, the nucleic sequence of the primer 401 is controlled for lack of sequence homology with all other hairpin probes and polymerase reagents. Preferably, the primer has a length of 18-20 nucleotides and a melting temperature of 59° C.+/−2° C. The primer 401 is used to initiate incorporation of at least one labelled nucleotide by a polymerase. Upon the association of the target-specific sequence and the target molecule, the arm 5′ of the target-specific sequence and the primer are accessible to the reagents of a polymerase reaction. The polymerization reaction is performed without denaturation of DNA and at 37-60° C. depending on the polymerase used. Suitable polymerases are DNA polymerase I klenow fragment from E. coli, thermostable Taq polymerases, T4DNA polymerase, and all available native and genetically engineered, excepting hot-start polymerases (Sambrook, J. et al. 1989. Molecular Cloning: A Laboratory Manual, 2nd Ed., Cold Spring Harbor Laboratory, Cold Spring Harbor, 5.44-5.47; Ausubel, F. M. et al. 1997. Current Protocols in Molecular Biology, vol. 1. John Wiley & Sons, Inc., New York, 3.5.11-3.5.12).

FIG. 4B shows such a hairpin probe in an open conformation wherein the arm 402 and the primer 401 that are 5′ of the target-specific sequence are accessible to participate in a polymerase reaction. A “reporter” molecule 403 that is complementary to a portion of the arm 402 and the primer 401 can then hybridize with those sequences. FIG. 4C shows such a hybridization of an arm 402, a primer 401 and a “reporter” molecule 403. The “reporter” molecule 403 may be complementary to a portion or the entire arm 402. However, the “reporter” molecule 403 must not be the complementary of the entire primer 401. The portion of the “reporter” molecule 403 that complements the portion of the arm 402 and the primer 401 must not associate with, or have nucleotides that complement to a portion of the 3′ end of the primer 401. Preferably, the nucleotide sequence of the “reporter” molecule 403 is 1 to 5 nucleotides shorter than the primer 401 to which it hybridizes. This attribute is shown by the non hybridized sequence 404 of the primer 401 and will allow a polymerase to elongate the “reporter” molecule 403 by adding nucleotides that are complementary to the primer 401. When a polymerase 405 does elongate the “reporter” molecule 403 (see FIG. 4D), labelled nucleotides 406 can be incorporated to generate a signal for detection of the hybridization of the target-specific sequence 407 with the target molecule 408.

Universal Addressing System

The present invention is also directed to a system wherein non-immobilized hairpin probes may be used in combination with immobilized hairpin probes. Designated a universal addressing system (“UAS”), this ex vivo screening system provides the advantage of a pre-made array of immobilized hairpin probes or linear probes which is adaptable for use with user-made non immobilized hairpin probes, thus sparing the end-user the task of biochip construction. For example, the UAS may provide a universal biochip in which the target-specific sequences of immobilized hairpin probes comprise a predetermined set of hairpin probes designed to associate with non-immobilized hairpin or linear probes that have specific sequence tags. This allows for the construction of a single biochip for any test condition. Target nucleic acids to be detected are first captured by the target-specific sequences of the non-immobilized hairpin or linear probes which, in turn, are designed to associate with immobilized hairpin probes via the specific sequence tag.

In a preferred embodiment, the UAS comprises two sets of hairpin probes. One hairpin probe, designated a “first hairpin”, is not immobilized on a substrate and has a tag extending from its 3′ or 5′ arm. The tag may hybridize with a single stranded nucleic acid sequence of 6 to 30 nucleotides or with a hairpin probe, designated a “second hairpin,” that is immobilized on a substrate, and may be partially or totally complementary to the tag. When the immobilized moiety is a second hairpin, the target-specific sequence of the second hairpin may be complementary to the tag sequence, a portion of the tag sequence and/or a portion of the tag sequence and a portion of the arm adjacent to the tag. The use of second hairpin probes that have target specific sequences for a portion of the tag and a portion of the arm adjacent to the tag will ensure that only first hairpin probes that have their target-specific sequence associated with a target molecule are able to hybridize with the second hairpin probe. If different tag sequences are to be used with the UAS and need to be discerned, a difference of at least 2 nucleotides, placed in the centre of the loop of the second hairpin may be required so that there is enough sequence differences with other tags to be specifically hybridized on its complementary target-specific sequence on the immobilized hairpin probe.

The invention is thus directed to an unlabelled universal addressing system comprising:

(a) at least two unlabelled linear first probes comprising
(i) a target-specific sequence that is 6 to 30 nucleotides in length; and
(ii) a “tag” sequence that is 10 to 50 nucleotides in length connected to the 5′ end of target specific sequence,
   wherein the target-specific sequence is not complementary with any other portion of said unlabelled probe; and

(b) at least two second probes capable of forming hairpin comprising:
(i) a sequence specific to a second target that is 6 to 30 nucleotides in length;
(ii) a second first arm of said second probe that is less than 10 nucleotides in length and is 5′ of said target-specific sequence;
(iii) a second second arm of said second probe that is less than 10 nucleotides in length and is 3′ of said target-specific sequence; and
(iv) a linker connecting the first or second arm to the substrate.
   said sequence specific to said target of the second probe is not complementary with any other portion of the said second probe; said first arm and said second arm of said second probe are perfectly complementary to each other; and further wherein said tag sequence of one of the first probes is complementary with said second target sequence of one of the second probes.

Preferably, the invention is directed to an unlabelled universal addressing system comprising:

(a) at least two first unlabelled probes capable of forming hairpin comprising:
(i) a target-specific sequence that is 6 to 30 nucleotides in length; and
(ii) a first arm that is less than 10 nucleotides in length and is 5′ of said target-specific sequence;
(iii) a second arm that is less than 10 nucleotides in length and is 3′ of said target-specific sequence; and
(iv) a “tag” sequence that is from 10 to 50 nucleotides in length, connected to the first arm or the second arm.
   wherein the target-specific sequence is not complementary with any other portion of said unlabelled probe; further wherein the first arm and the second arm are perfectly complementary to each other, and

(b) at least two second probes capable of forming hairpins, comprising:
(i) a sequence specific to a second target that is 6 to 30 nucleotides in length;
(ii) a second first arm of said second probe that is less than 10 nucleotides in length and is 5′ of said target-specific sequence;
(iii) a second second arm of said second probe that is less than 10 nucleotides in length and is 3′ of said target-specific sequence; and
(iv) a linker connecting the first or second arm to the substrate.
   said sequence specific to said target of the second probe is not complementary with any other portion of the said second probe; said first arm and said second arm of said second probe are perfectly complementary to each other; and further wherein said tag sequence of one of the first probes completed with arm sequence connected to this tag is complementary with said second target sequence of one of the second probes.

In a preferred embodiment, the target specific sequence of first hairpin probes is 10 to 25 nucleotides in length, and more preferably is 15 to 20 nucleotides.

Preferably, the target specific sequence of second hairpin probes is 10 to 25 nucleotides in length, and more preferably is 15 to 20 nucleotides.

The invention is also directed to an unlabelled universal addressing system wherein all first arms have an identical sequence.

It is further directed to an unlabelled universal addressing system wherein all first arm of second hairpin probe have an identical sequence.

The invention is also directed to an unlabelled universal addressing system wherein the same reporter molecule can be hybridized to the first probe or the second probe of the invention.

First arm of second probe of the invention and second arm of second probe of the invention may dissociate when a portion of the “tag” and a portion of the first or second arm connected to this tag are associated with target-specific sequence of second probe.

A specific embodiment of the invention is directed to an unlabelled universal addressing system wherein the “tag” associates with target-specific sequence of second probe.

FIG. 5 depicts how one embodiment of the UAS may function. In this embodiment, the target-specific sequence of the second hairpin probe is designed to hybridize with the tag and a portion of the arm adjacent to the tag of the first hairpin probe. FIG. 5A shows a UAS 501 with no target molecule present. The first hairpin probe 502, which has a tag 503, and the second hairpin probe 504, which is attached to a substrate 505 by a linker 506, are in a closed conformation. FIG. 5B shows how the first hairpin probe 502 assumes an open conformation upon association with its target 507 and a labelled “reporter” molecule 508. FIG. 5C shows the first hairpin probe 502 with its tag 503 and a portion of the arm 509 that is adjacent to the tag hybridized with the target-specific sequence 510 of the second hairpin probe 504. The location of the target molecule may then be determined by detecting the “reporter” molecule's location on the substrate.

Nucleic Acids Detection

The invention is also directed to a method of nucleic acid detection comprising:
(a) contacting ex vivo a nucleic acid sample with a biochip comprising at least two probes of the invention; and
(b) detecting a signal from at least one said probe of the biochip which has assumed an open conformation following contacting in step (a).

Detecting a signal in step (b) is preferably obtained by using a “reporter” molecule labelled with a detectable marker. Preferably, detectable marker may be a nucleotide analog, a fluorescent label, biotin, imminobiotin, an antigen, a cofactor, dinitrophenol, lipoic acid, an olefinic compound, a polypeptide, an electron-rich molecule, an enzyme, or a radioactive isotope.

In a preferred embodiment, nucleic acid sample of step (a) is already labelled with a detectable marker.

Preferably, all perfect hybrids formed upon association of target-specific sequences with the target molecules have a melting temperature equal within a range of 4° C., more preferably within a range of 1° C. The difference between melting temperature of hybrid formed upon association of the target-specific sequence with the target sequence, and a second melting temperature of hybrid formed upon association of the target-specific sequence with a molecule for which the target specific sequence is not designed is greater or equal to 5° C., more preferably 8° C.

Mutation Analysis

The present invention provides a method to design and use biochips of hairpin probes for analysing multiple DNA sequences that may present allelic variations. Particularly, this invention provides a tool for haplotype determination on short DNA segments presenting multiple single nucleotide variations on one sequence of DNA. Mutations analysable by this method include single nucleotide transitions (or substitutions), deletions or insertions of a single nucleotide or multiples variations separated by a short sequence as well as large insertions and deletions. The analysis of such mutations may need more than one allele-specific probe. Up to four allele specific probe may be designed and immobilised for one single transition analysis, depending on the degree of allelism observed for each genetic variation, and the confidence needed for the analysis. TABLE I gives, for example the sequences of hairpin probes that may be used for analysing of the transition found in the codon 142 of TCF 1 gene, which encodes the hepatic nuclear factor 1 protein (“HNF1a”) (Linder, T. et al. 1999. Hum. Molec. Genet. 8:2001-8), 142CC CY5, which is the Cy5-labelled target molecule for StrNCb hairpin probe, and 142CT CY5, which is the Cy5-labelled target molecule for StrNTb hairpin probe. The bold characters represent the loop sequence, whereas the underlined characters indicate the position of the mutation site. The naturally occurring mutation is a C/T transition, or a G/A transition on the complementary strand. The hairpin probes that need to be used for analysis of this possible mutation are the probe bearing a C, StrNCb, and the probe bearing a T, StrNCT. The other two hairpin probes, StrNGb and StrNAb, may be used but are not required.

TABLE I

| | | |
|---|---|---|
| Name | Sequence | SEQ ID No |

| | | |
|---|---|---|
| StrNCb | 5′- GCG AGC CAA CCA GTC CCA CCT GTC GCT CGC -3′ | 1 |
| StrNTb | 5′- GCG AGC CAA CCA GTT CCA CCT GTC GCT CGC -3′ | 2 |
| StrNGb | 5′- GCG AGC CAA CCA GTG CCA CCT GTC GCT CGC -3′ | 3 |
| StrNAb | 5′- GCG AGC CAA CCA GTA CCA CCT GTC GCT CGC -3′ | 4 |
| 142CC CY5 | Cy5 - 5′- AGG TGT TGG GAC AGG TGG GAC TGG TTG | 5 |
| | AGG CCA GTG GTA TCG -3′ | |
| 142CT CY5 | Cy5 - 5′- AGG TGT TGG GAC AGG TGG AAC TGG TTG | 6 |
| | AGG CCA GTG GTA TCG -3′ | |

Using a precisely mapped genomic sequence, all polymorphism variations within the studied region are referenced. For variations that need to be analyzed for allelic determination, a set of PCR primers may be designed using the general guidelines known by one skilled in the art. To avoid primer sequences that may incorporate allelic variants and to maximize the specificity and yield of PCR reactions, attention is paid not to design primer pairs in genomic region containing known polymorphisms. For each mutation that requires an allelic determination, at least two hairpin probes are designed and used. The position of the allelic variation is localized as closely as possible to the centre of the specific loop sequence (see 101 in FIG. 1) but may vary depending on the sequence characteristics. When allelic variant position is localised far from centre of the loop, Tm of hybrid formed upon association of target-specific sequence and a target molecule not fully complementary to target-specific sequence decreases.

When designing hairpin probes for use in the same analysis, target-specific sequence length modification (see supra) is employed, in order that all Tm of hybrids between target specific sequence and target molecule are equal, more or less 1-4° C. For example, the allelic variation to be detected may be from 1 to 10 bases, preferably from 2 to 5 bases, from the centre of the loop. Once the target-specific sequences of all of the hairpin probes are designed to have the Tm in an acceptable range (see supra), the Tm of each loop hybridized with the other alleles (heteroduplexes) is then calculated using Meltcalc (see supra). The variation of Tm between perfect and non perfect duplexes of DNA is kept as large as possible, and is considered to be sufficient for adequate discrimination when Tm of hybrid formed upon association of target-specific sequence and a target molecule not fully associated, i.e. hybrid possessing at least one mismatched base, is at least 5° C. below Tm of hybrid between target-specific sequence and target molecule. More preferably, Tm of hybrid formed upon association of target-specific sequence and a target molecule not fully associated is at least 8° C. below Tm of hybrid between target-specific sequence and target molecule.

Target sequences may be genomic DNA, or labelled or unlabelled DNA sequences amplified by PCR. The size of the assayed sequence in terms of nucleotide number may be the same as the loop part of the hybridization probes, but a larger number of nucleotide is preferred with no limitation in term of maximum size of the assayed sequence. Each strand of these double stranded target sequences may be thermically separated and hybridized as individual species or as a mixture with the biochip of hairpin probes so that only the perfect matched sequence is hybridized to its specific immobilized hairpin probe.

The relative abundance of each detected allele in one sample may be determined by measuring the relative signal for each probe specific for each allele. For example, for a single bi-allelism corresponding to a transition, two out of four hairpin probes (each with one different base corresponding to one allele) should give a signal for a heterozygous sample. That allele may be determined by the position of the hairpins on the solid surface. For a homozygous sample, only one out of the four hairpin probes should give a signal.

The invention is thus directed to a method of detecting a genetic variant ex vivo in a nucleic acid sample comprising:

(a) contacting the sample with a biochip of probes of the invention comprising at least two probes, wherein at least one probe of the biochip is a genetic variant specific probe of the invention possessing a loop fully complementary with genetic variant, and

(b) detecting a signal from the hairpin probe of the genetic variant,

the signal detected in step (b) indicating the presence of the genetic variant in nucleic acid sample.

Preferably, the detected genetic variant is a single nucleotide polymorphism, a deletion or insertion of one or more nucleotide, or a duplication of one or more nucleotide.

In a preferred embodiment, a “reporter” molecule may hybridise with each hairpin probe of the biochip. Detecting a signal in step (b) is preferably obtained by using a “reporter” molecule labelled with a detectable marker. Nucleic acid sample of step (a) may be previously labelled with a detectable marker.

Sequencing

The present invention provides a method for ex vivo DNA sequencing oligonucleotides samples, avoiding manual preparation steps of sequencing methods using chemical degradation and chain termination with di-deoxy nucleotides. The invention comprises design and use of hairpin probes and biochips of hairpin probes in which each probe's target-specific sequence comprises a sequence identical to at least a portion of another target-specific sequence of another probe that is immobilised on the same biochip. Depending on the size of the sequence to analyse, target-specific sequences of hairpin probes immobilised are further designed to hybridise with any oligonucleotide sequence which length is at least equal to the length of target-specific sequence of hairpin probes. By this method, a biochip can be designed for sequencing all oligonucleotides of predetermined length. Detection of the hybridisation that occurs between portion of the sample and hairpin probes may be monitored, and the sequence determined according to present invention descriptions, and those of U.S. Pat. Nos. 6,270,961; 6,025,136; 5,871,928; and 5,695,940.

The invention is thus directed to a method of ex vivo sequencing an oligonucleotide comprising:

(a) contacting the sample containing the oligonucleotide with a biochip comprising at least two probes of the invention; and

(b) detecting a signal from at least one of said probes of the biochip;

the signal detected in step (b) being used in determining the sequence of the oligonucleotide.

In a preferred embodiment, a “reporter” molecule may hybridise with each hairpin probe of the biochip. The detection in step (b) is preferably obtained using one reporter molecule labelled with a detectable marker. The sample in step (a) may be previously labelled with a detectable marker.

Gene Expression Analysis

The present invention provides a method to design and use hairpin probes of the invention and hairpin probe biochips of the invention for analyzing gene expression.

This embodiment may be useful for differential expression analysis of unlabelled nucleic acids when a large number of different sequences need to be analyzed in term of relative abundance. For each gene that may be analyzed, at least one hairpin probe may be designed for each exonic part of the gene.

The invention is also directed to a biochip of probes of the invention comprising at least two probes of the invention immobilised on a substrate. In a preferred embodiment, a stem structure is identical for each of at least the two hairpin probes. More preferably, a “reporter” molecule may hybridise with each probe of the invention of the biochip.

In order to monitor expression levels from cells extracts or tissues samples, total RNAs, mRNAs, or labelled or unlabelled cDNAs and cRNAs may be used as the target sequence for the hairpin probes or the hairpin biochips. If cDNAs and cRNAs are used for expression analysis, those target sequences may be labelled by fluorescent dyes incorporated during reverse transcription, or additional PCR steps.

For differential expression analysis, various conditions may be compared. These conditions may be physiological stress or differentiation stages, activation of a metabolic pathway, or transcriptional activity of a drug. For example, each of the two pools of nucleic acids such as unlabelled mRNAs or total RNAs extracted from cells or tissues or cDNAs may be treated separately for hybridization with a hairpin biochip specifically designed for each condition or with a set of specifically designed first hairpin probes for analysis with the UAS.

Detection of Alternative Splice Products and Measurement Conditions for Relative Quantities

This invention provides a method for analyzing alternative splice products and determining relative abundance of each alternative splice product in different physiological states for the same tissue, or relative abundance of each alternative splice product in different tissues for the same gene. This application may be useful for analysis of multiple variants of many genes whose products are thought to interact in a defined pathway.

The invention is thus directed to a method of detecting ex vivo an alternative splice product of a gene in a nucleic acid sample comprising:
(a) contacting the sample with a biochip of the invention comprising at least two probes of the invention, wherein at least one said biochip probe is a probe specific for an exon of the gene or specific for a junction of two exons; and
(b) detecting a signal from the exon-specific probe or two exons junction-specific probe, the signal detected in step (b) indicating the presence of the alternative splice product of the gene in the nucleic acid sample.

Preferably, the nucleic acid sample comprises mRNA.

The target-specific sequences of hairpin probes are designed to be complementary to mRNA or cDNA sequences. More particularly, they are designed to be complementary to the various splicing products already known of each target sequence. A single nucleotide deletion as well as large deletions resulting in various splice variants may be detected on the same biochip of hairpin probes specific to these variations.

Each putative or known exonic sequence of a studied gene may have a specific hairpin probe designed for detecting the presence or absence of the exon, or portion of exon or junction between two exons, in the gene product. The design of the hairpin probes is performed in a similar manner as that for gene expression analysis, and may use UAS to address sequences on a substrate. Moreover, variations in length or in composition of the mRNA or cDNA analysed, may be analysed by several hairpin probes, which target-specific sequences are designed to hybridise with the centre of analysed region. The analysis of deletion or substitution of a nucleotide between two mRNA, may be analysed by e.g. designing two hairpin probes having one nucleotide difference localised in the centre of target-specific sequences.

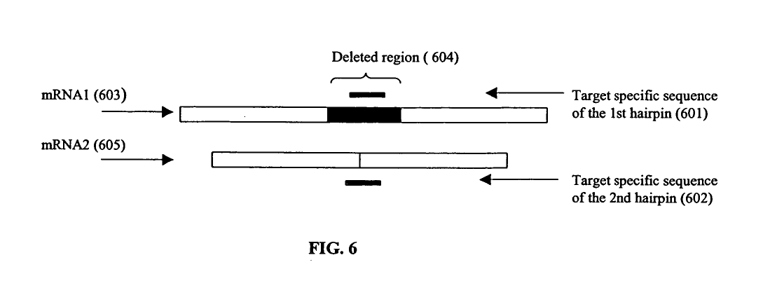

The design of these probes is similar to that of probes designed for mutation analysis. For gene detection of large deletions in mRNA, target-specific sequences of two hairpin probes 601 and 602 may be designed to hybridize to a mRNA sequence (FIG. 6). The target-specific sequence of the first hairpin probe 601 may be designed to hybridize to a region 604 of an mRNA 603, that may be deleted, and be an alternative splice form. The target-specific sequence of second hairpin probe 602 may be designed to detect alternative splice form 605 by hybridizing to the regions just 5′ and 3′ of the deleted region 604. The design of these probes is similar to that of probes designed for expression analysis, and a biochip may then be used to screen for the presence or absence of transcribed exons of the same gene.

The procedure for differential analysis of splicing variants is the same as for differential expression analysis. mRNAs derived from two tissues in a physiological state or from two physiological states of one tissue or from two different tissues are separately retro-transcribed into cDNAs and labelled (a different label for each tissue or state). Labelled cDNAs from the two sources are mixed and hybridized with hairpin probes biochips or hybridized with hairpin probes of the UAS system, and further hybridized with biochips of second hairpin.

In a preferred embodiment of the invention, a “reporter” molecule may hybridise with each hairpin probe of the biochip. The detection in step (b) is preferably obtained using one reporter molecule labelled with a detectable marker. The sample in step (a) may be previously labelled with a detectable marker.

Hairpin Probes for Detection of Molecules of Foreign Origin

This invention may be used for detection of molecule from any nucleotide containing organism, or any remnant thereof such as, but not limited to, pathogens, micro organisms, viruses, parasites, or a genetic modification as found in genetically modified organisms (“GMO”) via detection of DNA, RNA, or any other molecules that would associate with a hairpin probe. According to this invention, “any remnant of any nucleotide containing organism” means any molecule, or any molecule resulting from that molecule, from a nucleotide containing organism that has entered into another cell, for example, but not limited to, viral DNA in an infected cell or mRNA produced from that viral DNA. This application may be particularly useful in situations where different molecules of foreign origin have to be detected in the same sample, or to analyze genetics variants in a biological sample.

The invention is also directed to a method of detecing ex vivo any nucleic acid containing organism or remnant thereof comprising:

(a) contacting a nucleic acid sample with a biochip comprising at least two probes of the invention, wherein at least one of said probe is specific for a nucleic acid containing organism or a remnant thereof; and

(b) detecting a signal from the probe specific of nucleic acids of the organism,

the signal detected in step (b) indicating the presence of the nucleic acid containing organism or a remnant thereof.

The nucleic acid containing organism is preferably a virus or a bacterium.

In a preferred embodiment of the invention, a “reporter” molecule may hybridise with each hairpin probe of the biochip. The detection in step (b) is preferably obtained using one reporter molecule labelled with a detectable marker. The sample in step (a) may be already labelled with a detectable marker.

In a preferred embodiment, total RNAs, mRNAs, or labelled or unlabelled cDNAs as well as labelled or unlabelled cDNA amplified in PCR reactions may be used on biochips of hairpin probes for detecting presence or absence of the considered pathogen, micro organism, virus, or a GMO. For each molecule of foreign origin to be analyzed, at least one hairpin probe is designed for at least one exonic part of genes known to be expressed at consistent levels in the organism being screened.

Loss of Heterozygosity and Allelic Imbalance Analysis

The invention is further directed to a method to design and use probes of the invention, and biochips of probes of the invention for analysing allelic imbalances caused by deletions or insertions of chromosomal fragments as well as loss of heterozygosity.

This application may be useful for analysing chromosomal deletions in which chromosomal breakpoints are known or unknown. Those breakpoints in a DNA fragment, where the deletion is located, are frequently unknown, and sometimes specific to an individual. (Mateo M. et Al. (1999), AM. J. Path., 154(5), 1583-1589). This type of genetic alteration is found in cancer like prostate, breast, and some types of colon cancer as well as some lymphomas. (Larsson C. M. et Al. (2001), Molecular Diagnosis., 6(3), 181-188).

The present invention is directed to genomic sequences comparison between healthy cells (e.g. blood lymphocytes) with presumed tumoral cells (e.g. cells from biopsies, or fluids like urine, cephalo-rachidian fluid or secretions) by using a biochip of probes of the invention as analysing means, and a differential analysis method.

This method of analysis consists in amplifying microsatellite sequences with PCR, using methods well known in the art, and to hybridise PCR products on a biochip of the invention. Microsatellite sequences are highly polymorphic repeated sequences, present in the genome at regular intervals, and flanked with sequences suitable for PCR primers positioning (Goldstein, D. and C. Schlotterer, eds. 1999. Microsatellites: evolution and Applications. Oxford University Press). Amplified microsatellite sequences are chosen in the putative deletion region, in order to have these regions amplified in absence of deletion, and to lack PCR product amplification when the deletion is present (for selecting microsatellite sequences, and suitable PCR primers: Genome Database, http://www.gdb.org). In the present invention, microsatellite sequences of healthy and presumed tumoral cells are individually amplified and labelled by using PCR primers differently labelled for each of the two types, healthy or tumoral. Preferably, a multiple PCR amplification of microsatellite markers (multiplex PCR) may be used. From one to several microsatellite sequences may be amplified depending on the pathology and the analysis to be performed. A preferred labelling is the amplification of microsatellite from healthy cells with one PCR primer labelled with Cy3, and an unlabelled complementary primer, and the amplification of microsatellite from tumoral cells with one PCR primer labelled with Cy5, and an unlabelled complementary primer. PCR products from each microsatellite markers, Cy3 labelled for healthy cells, and Cy5 labelled for tumoral cells are then treated to discard free PCR primers (not incorporated in PCR) remaining after amplification. Several methods to discard PCR primers are envisioned, with a preferred method using exonuclease I and shrimp alkaline phosphatase (exo/sap), according to protocol of the kit exo/sap “ExoSAP-IT” (USB Corporation, 26111 Miles Road, Cleveland Ohio 44128, USA).

Following this treatment, amplification products from microsatellite sequences are pooled in one solution, and hybridised on a biochip of probes of the invention. This pooling step may be performed alternatively before the PCR primers elimination step. The biochip of present invention comprises at least one probe. Such probe is at least partially complementary with one of the PCR primers used for amplifying microsatellite sequence, and in a preferred embodiment, fully complementary with a region or the entire sequence of primer. Several types of probes may be used on the biochip of the invention, or preferably, hairpin probes of the invention modified by addition of a linker, or probes of the UAS system. The preferred method probe sequence is designed using present invention characteristics, in particular when several probes are used with several microsatellites sequences for analysis, Tm of all hybrids between target specific sequence of the probes, and complementary target microsatellite sequences are equal within a range of 4° C., preferably within a range of 1° C. Likewise, when several probes are used on a biochip, one of the arms of all the probes of the preferred method can hase the same sequence. When several microsatellite sequences are analysed, if the presence of more than one probe is needed on the biochip, those probes may be of several type, i.e. linear, and/or hairpin, and/or probes of the UAS system.

In a preferred method, probes of the UAS system are used to add amplification negative standard during PCR of microsatellite sequences. These negative standards are obtained by amplifying a microsatellite sequence in the absence of genomic DNA from healthy or tumoral cells, but in presence of reagents needed for PCR and a pair of primers, at least one of them being a first linear probe of the UAS possessing a “tag” sequence fully complementary to target-specific sequence of the second immobilized probe.

When a PCR product obtained by amplifying a microsatellite sequence of healthy and presumed tumoral hybridises with a probe of the biochip, the biochip's probe hybridises with primer sequences of the PCR localised at the ends of PCR products. Differential labelling of amplicons from cells with Cy3 and Cy5 produces for each probe of the biochip a signal composed of emission for Cy3, due to hybridization of healthy cells PCR product with probe, and emission for Cy5, due to hybridization of presumed tumoral cells PCR product with probe. By measuring the relative emission part for each of the labels, one can deduce the relative quantity of microsatellite sequence-specific PCR products from healthy and tumoral cells. Microsatellites sequences being not amplified when a deletion occurs in the microsatellite containing region, a decrease of Cy3 emission (presumed tumoral cell) is observed, compared to Cy5 emission (healthy cell) when deletion has occurred.

In this invention, the analysis of relative abundance of PCR products for amplification of a microsatellite sequence is obtained after hybridisation, by comparing relative emission values for Cy3 and Cy5 with an internal calibration curve. This calibration curve is obtained by amplifying and competitive hybridisation of DNA from non-deleted microsatellite markers located on the Y chromosome (heterozygosity markers) with the probes of the biochip of the invention, and other non-deleted markers located on other chromosomes (homozygosity markers). In the preferred embodiment, amplification of these markers is performed with both types of samples (healthy and presumed tumoral cells) in the same time as the amplification of the other microsatellite markers, using differential labelling with Cy5 and Cy3 as described before. DNAs used to amplify heterozygosity and homozygosity markers may be issued from DNA extraction of healthy and tumoral cells of the individual, or extemporaneous preparations of this DNA from male individuals, of known microsatellite markers genotypes (having no deletions for these markers) tested in this amplification.

Alternatively, the calibration curve may be constructed by PCR amplification of two or more microsatellite markers that are not deleted for considered pathology. Those markers are amplified in presence of a serial dilution of DNA extracted from each cellular type that are compared (healthy, and presumed tumoral cells), and in the same time as the amplification of the other microsatellite markers. After performing the analysis, the calibration curve is obtained by reporting on a graph, the x-axis of which is Cy3 fluorescence intensity, and the y-axis of which is the Cy5 fluorescence intensity, respective values of both fluorescence emissions for each of the biochip probes corresponding to markers' calibration.

The object of this invention is also directed to Kits of biochips from the invention, to probes and reagents including RT-PCR reagents for allelic imbalances analysis in:
prostate cancers, kidney cancers and bladder cancers
breast cancers
colon cancers
lung cancers
analysis of presence of large deletions in hereditary diseases like Duchenne myopathy, and acquired diseases.

### BRIEF DESCRIPTION OF FIGURES

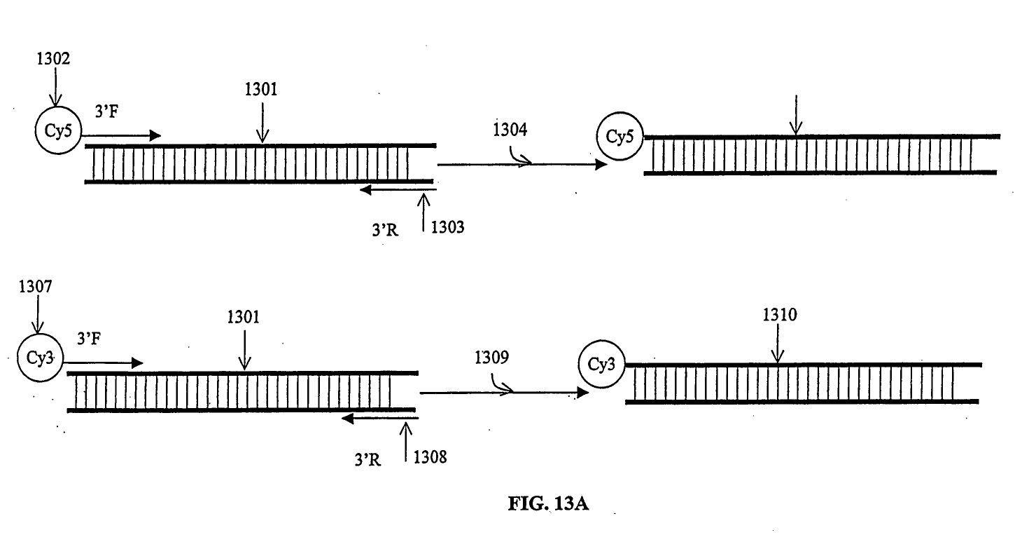

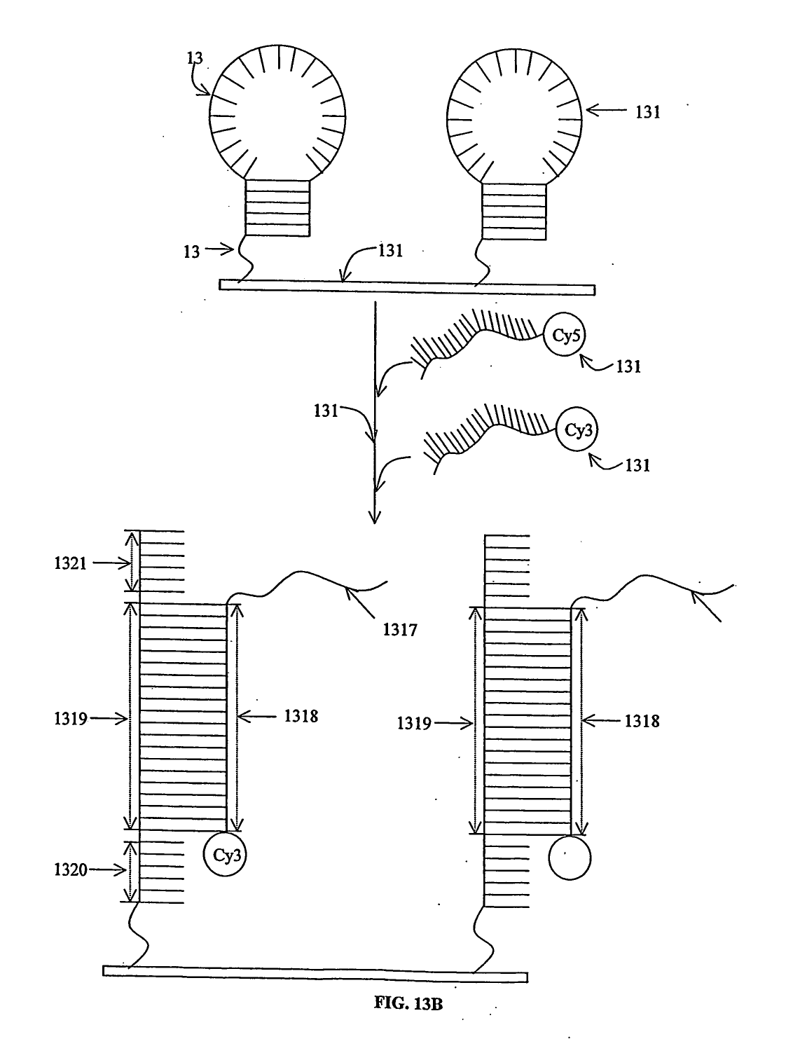

FIG. 13A: PCR primer 1302, labelled at its 5′ end with a fluorescent label (Cy5), and located 3′ end of sense strand of microsatellite sequence 1301, is used with unlabelled primer 1303, which is 3′ end of anti-sense strand of microsatellite sequence 1301, to amplify this microsatellite sequence in genomic DNA samples extracted from healthy cells 1304, and to produce the PCR product 1305.

The PCR primer 1307, labelled at its 5′ end with a fluorescent label (Cy3), and located 3′ end of sense strand of microsatellite sequence 1301, is used with unlabelled primer 1308, which is 3′ end of anti-sense strand of microsatellite sequence 1301, to amplify this microsatellite sequence in genomic DNA samples extracted from presumed tumoral cells 1309, and to produce the PCR product 1310.

FIG. 13B: The probe biochip is contacted 1315 with the mixture containing PCR products from amplification steps 1304 and 1309 of FIG. 13A. The probe biochip is depicted with two hairpin probes 1311 and 1314, identic, both immobilised on substrate 1312 via a linker 1313. The target-specific sequence 1319 of these two probes is fully complementary with sense PCR primers sequence 1318, included in PCR products 1316 and 1317. PCR product 1316 is Cy5 labelled, and PCR product 1317 is Cy3 labelled. The PCR product 1316 is obtained upon denaturation of the PCR product 1305 (FIG. 13A), and the PCR product 1317, upon denaturation of the PCR product 1310 (FIG. 13A).

Hairpin Biochip Kits

The present invention further provides kits for use in detecting the presence of target molecules in a sample. Such kits typically comprise two or more components necessary for performing such an assay. Such components may include a biochip of hairpin probes and a supply of the required reagents for detecting selected target molecules. Alternatively, the kit may comprise immobilized and/or non-immobilized hairpin probes and reagents such as is used in the UAS. A preferred kit comprises a biochip of hairpin probes, a set of non-immobilized hairpin probes and/or a “reporter” molecule and one or more additional reagents. Another preferred kit comprises biochip of linear probes, a set of non-immobilized hairpin probes and/or a “reporter” molecule, and one or more additional reagents.

### EXAMPLES

### Hybridization of Target DNA with Immobilized Hairpin Probes

The following examples are presented by way of illustration of the present invention, and are not intended to limit the present invention in any way. In particular, the examples presented herein below describe use of hairpin biochips for detection of target sequences.

The Nucleic Acid Molecules

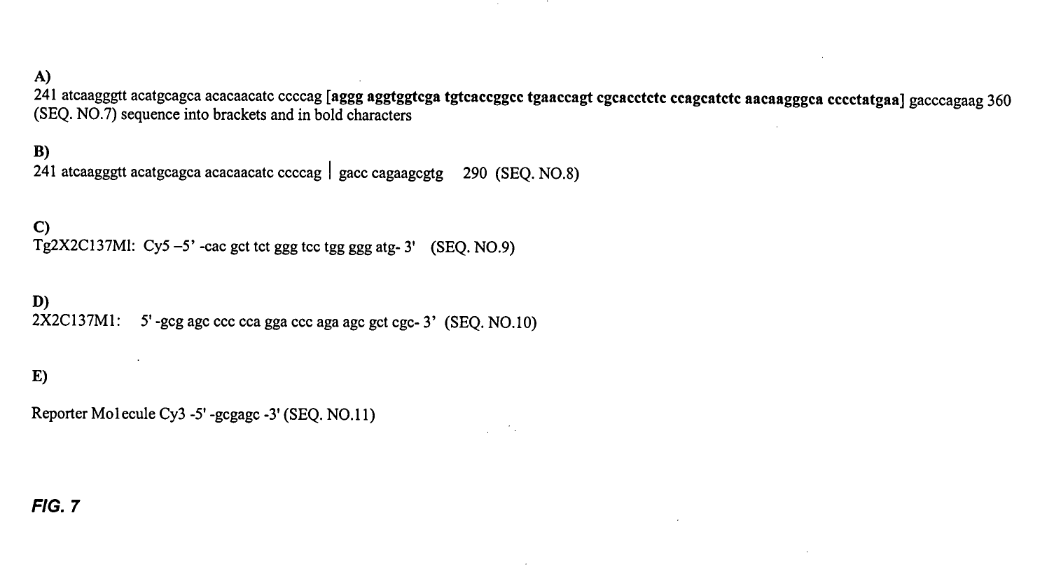

The codon 137 mutation in the exon 2 of the TCF2 gene is a 75 base pair deletion resulting in a Diabetes Type II phenotype with severe renal dysfunctions (Linder, T. et al. 1999. Hum. Molec. Genet. 8:2001-8). The target sequence used in this example corresponds to positions 266 to 290 of the deleted sequence of codon 137 in the exon 2 of the TCF2 gene. FIG. 7A shows the location of the codon 137 mutation of exon 2. The bold bracketed portion, bases 276 to 350, represents the deleted portion in mutated individuals. FIG. 7B shows the sequence of exon 2 when the mutation occurs.

The target sequence, Tg2X2C137M1, used in this example, FIG. 7C, is the complementary sequence, or negative strand, of the bold sequence in FIG. 7B. The target-specific sequence of hairpin probe 2X2C137M1, shown in FIG. 7D, was designed by selecting 18 nucleotides in the middle of the mutated target sequence, which is a perfect complement to the target sequence. The “reporter” molecule designed for the following studies is shown in FIG. 7E and has a melting temperature of 23° C. using a 1M sodium salt and 1 μM oligonucleotide solution. The stem structure of the 2X2137M1 hairpin probe has a Tm of 72° C. The target-specific sequence of the hairpin probes were designed to have a Tm for the hybrid formed between target-specific sequence and the target molecule of 60° C.±2° C. with Meltcalc software parameters set-up at 100 mM sodium and oligonucleotide concentration at 100 mM (Tm for Tg2X2C137M1/2X2C137M1 duplexes=60.8° C.).

Meltcalc software was used for thermodynamic melting point prediction of oligonucleotide hybridization. This software allows setting a Tm through variations in salt concentrations, DMSO, and oligonucleotide concentrations. The hairpin probes of these examples were designed using 0.1 mM Na salt and 0.1 mM oligonucleotide concentrations. The association on each arm of hairpin probes with itself is assessed with mfold, the parameters for which are a folding temperature of 37° C., 1M sodium, no magnesium, oligomer correction type, five percent suboptimality, and an upper bound of computed folding of 50. All hairpin probes fed into mfold have a maximum continuous stem length of 6 nucleotides with a Tm of 65-72° C. using the above parameters. The Tm for perfectly complementary hybrids between target molecules and target-specific sequences of wild type and mutants is 60° C., with a DTm of at least 5° C. between perfect and imperfect hybridizations, and each stem has a Tm of 65-72° C.

Immobilization of Hairpin Probes

All hairpin probes, synthesized with an aminolinker on the 5′ end, were spotted on N-Hydroxy-succinimide (“NHS”) activated glass slides (NoAb Diagnostics, Mississauga, Ontario, Canada) with spotting buffer, which was provided with the slides, for 45 minutes at 30° C. in a humidity chamber. The remaining NHS groups were deactivated by reaction with a blocking solution, also provided with the slides, at room temperature for 30 minutes. The slides were then washed in citrate buffer pH 7, comprising 0.1 M sodium chloride, and rinsed with milliQ water.

Hybridization of the Target Sequence

Each biochip of immobilized hairpin probes was treated with 20 μL of a 400 nM solution of Cy5-labelled target sequence, Tg2X2C137M1 in 6×SSC buffer containing 50% formamide. A cover slip was placed on the top of each slide and the target sequence solution was left to hybridize at room temperature for one hour. Each biochip was then rinsed for five minutes in 4×SSC and then rinsed for two minutes in 0.1×SSC buffer. Finally, each slide was imaged in a biochip scanner (Axon Instrument, Inc., Union City, Calif., USA) using both Cy5 and cy3 excitation and emission wavelengths.

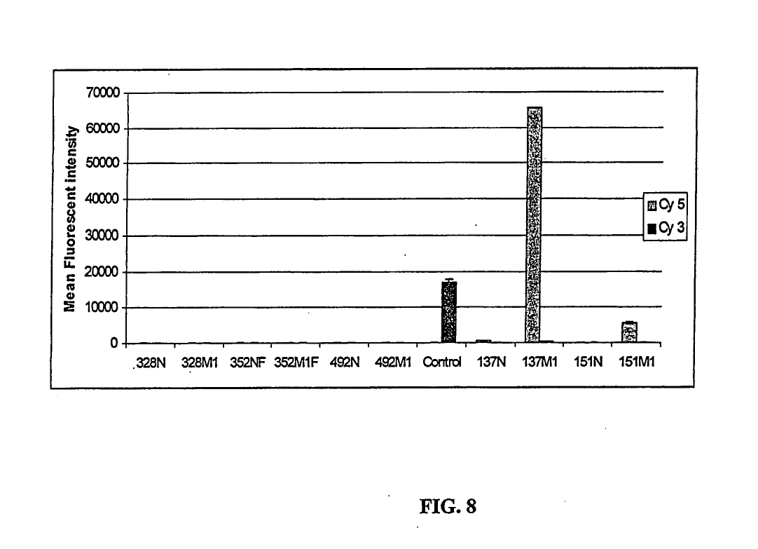

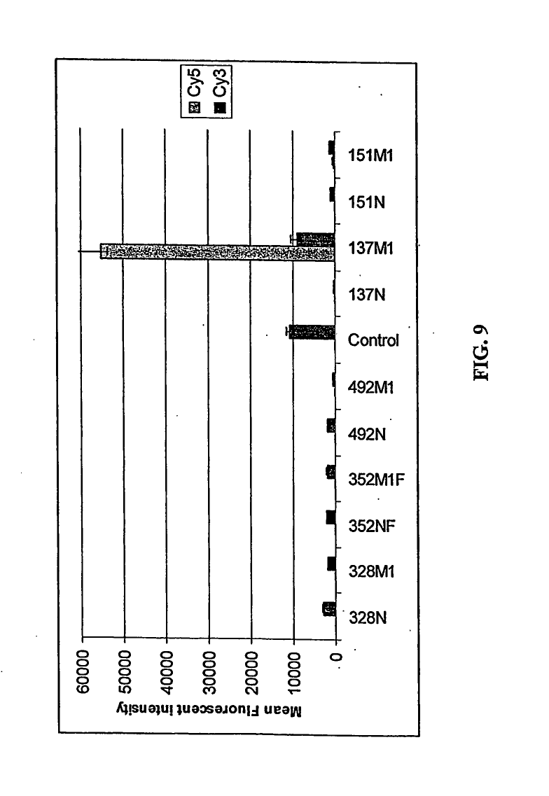

FIG. 8 shows Cy5 and Cy3 fluorescent intensities after hybridization of Cy5-labelled target sequence. Each value is a mean of five replicate spots of each hairpin probe. The ten hairpin probes used in this screening are designed for the analysis of five different mutations localized in different exons of the TCF2 gene. Only 2X2C137M1 (“137M1” in FIG. 8) is perfectly complementary to the target, Tg2X2C137M1. The control is a 5′-Cy3 labelled linear single stranded sequence modified on its 3′ end with an aminolinker. The control is used for the spot positioning and quality control of the spotting. Cy5 signal associated with hybridization of Cy5-labelled target sequence is observed essentially on spots corresponding to the target's perfectly complementary sequence, the loop of the 2X2C137M1 hairpin probe.

Hybridization of the Reporter Molecule

Following hybridisation with target molecule, each biochip of immobilized hairpin probes was treated with 20 μl of a 1 μM solution of Cy3-labelled “reporter” molecule in a 6×SSC buffer containing 50% formamide. A cover slip was placed on top of each slide and the “reporter” molecule solution was left to hybridize at room temperature for two hours. Each biochip was then rinsed for two times for five minutes in 4×SSC buffer and then rinsed for two minutes in 0.1×SSC buffer. Each slide was imaged in a biochip scanner using both Cy5 and Cy3 excitation and emission wavelengths.

FIG. 9 shows mean Cy5 and Cy3 fluorescent intensities hybridization of Cy3-labelled “reporter” molecule based on 5 replicates measures. Cy3 signal associated with hybridization of Cy3-labelled “reporter” molecule is observed essentially on spots where Cy5 labelled Tg2X2C137M1 sequence is hybridized with its perfect complement i.e. the 2X2C137M1 hairpin probe. The results show that the “reporter” molecule preferentially hybridises to opened probes and these open probes are only found when target molecule hybridize with the hairpin probes that contain the target-specific

Specificity of the Hairpin Probes

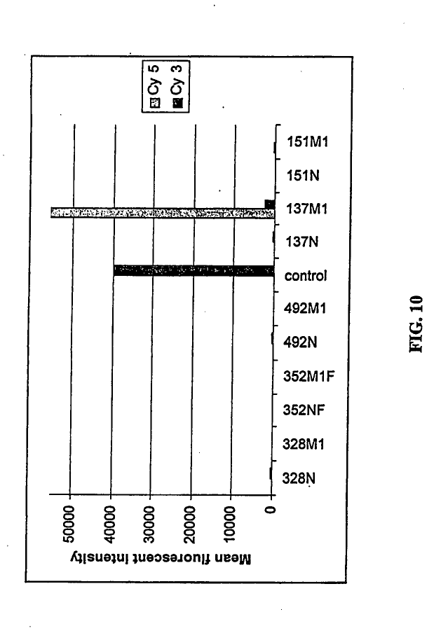

Non-specific binding between hairpin probes and target molecules may be reduced by varying the conditions of hybridization. In FIG. 8, non-specific interaction between the Cy5-labelled target and the 151M1 probe is clearly observed. In FIG. 9 evident interaction between the Cy3-labelled “reporter” molecule and all of the hairpin probes is observed, the 2X2C137M1 hairpin probe excluded. One way to remove non-specific interactions is with additional washes. FIG. 9 shows that with additional washes the non-specific binding between the Cy5-labelled target sequence and the 151M1 hairpin probe is reduced. Another way non-specific binding may be reduced is with the use of more stringent conditions. FIG. 10 shows how a more stringent washing for 15 to 30 minutes with 4×SSC buffer at 28° C., which is Tm of the “reporter”+5° C., removes most of the non-specific binding between the reporter molecule and all of the hairpin probes except for the 2X2C137M1 hairpin probe.

Single Nucleotide Discrimination with Hairpin Probes

TABLE II shows the sequences of sixteen hairpin probes that were designed to analyze mutations in the genes for hepatic lipase (“LIPC”), cholesteryl ester transferase protein (“CEPT”), lipoprotein lipase (“LPL”), and TCF1. Summarized in Table II is the name of each hairpin probe, correspondence with gene name and codon sequence of each hairpin probe, and the number and type of mismatches each hairpin probe has when hybridized with the target molecules. The target specific sequences of the hairpin probe are either perfectly matched, or complementary, to the specified target molecule; or not perfectly complementary, having one or two mismatches; or not complementary at all to the target molecule, as indicated with “−.” The hairpin probes were designed by selecting a loop sequence of 18 to 24 nucleotides complementary of the target sequence bearing a single nucleotide polymorphism. All SNPs variants were positioned in the centre of the loop (bold characters) with 9 to 11 nucleotides flanking the polymorphic position. The Tm of each wild-type and mutant SNP variant loop hybridized with its specific target sequence was designed to be 60° C.±2° C. using Meltcalc software with parameters set at 100 mM sodium and 100 mM oligonucleotide concentrations. The sequence of hairpins was adjusted by adding and/or removing one or more nucleotide on each end of the loop to reach the Tm of 60° C. with a variation in melting temperature between heteroduplexes and homoduplexes of at least 5° C.

The immobilization of the hairpin probes was performed as described above. Each biochip of immobilized hairpin probes was treated with 20 μL of a solution of 1M sodium buffer, 400 nM of Cy3-labelled M19 target, and 400 nM Cy5-labelled 142 CC for one hour in 6×SSC buffer at 20° C. while covered with a glass cover slip. The biochip was then rinsed for five minutes in 4×SSC and then washed in 0.1×SSC for two minutes. The biochip was then imaged as described above using both Cy5 and Cy3 excitation and emission wavelengths using a photomultiplier factor (“PMT”) of 600×600.

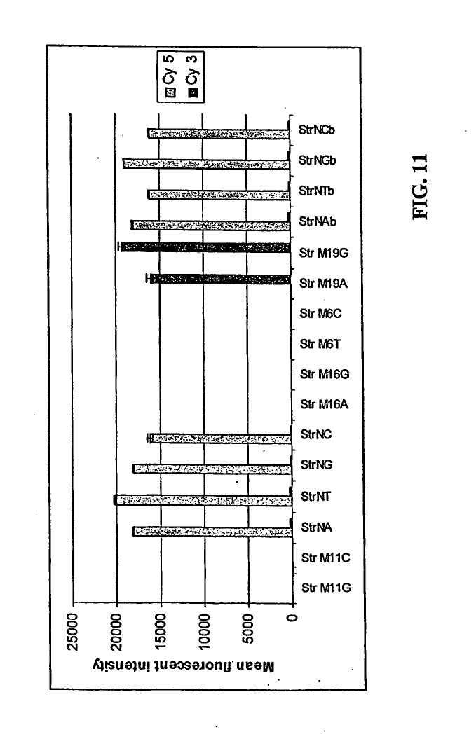

FIG. 11 shows the mean Cy5 and Cy3 fluorescent intensities for five replicates after hybridization of Cy5-labelled and Cy3-labelled target sequences. The Cy3 signal corresponds to hybridization of the M19 target and the Cy5 signal corresponds to the hybridization of the 142 CC target. Only hairpin probes partially or fully complementary to target sequences were able to generate a signal upon hybridization of the target sequences under low stringency conditions. To discriminate homoduplexes from heteroduplexes, each biochip was washed for 20 minutes in 6×SSC buffer at 50° C. followed by a rinse with 6×SSC and then re-scanned for Cy5 and Cy3 fluorescence at a PMT of 600×600.

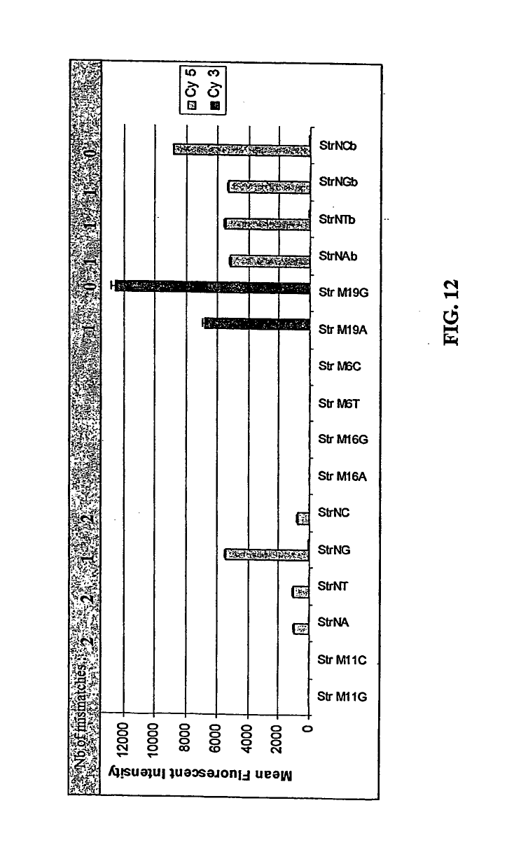

FIG. 12 shows the mean Cy5 and Cy3 fluorescent intensities for five replicates after subjecting each biochip to a more stringent wash. Hairpin probes that have perfect complementation with the labelled targets, StrM19G and StrNCb, have the highest hybridization. Hairpin probes that have a single mismatch have the next higher levels of hybridization and those probes that have two mismatches have the lowest hybridization levels. This differentiation in hybridization is obtained through the higher stability of perfect complementation between the hairpin probes and the target sequences.

TABLE II

| | | | | |
|---|---|---|---|---|
| Gene/Codon | | | | |
| Number in the | Probe | | Target | SEQ |
| gene | Name | Sequence | Mismatch | ID No |

| | | | | |
|---|---|---|---|---|
| LPL/ | StrM11C | 5′-GCG AGC GAA TAA GAA GTA | — | 12 |
| 477 S447X C/G | | GGC TGG TGA GC GCT CGC-3′ | | |
| | StrM11G | 5′-GCG AGC GAA TAA GAA | — | 13 |
| | | GTA GGC TGG TGA GC GCT | | |
| | | CGC- 3′ | | |
| LPL/ | StrM16G | 5′ -GCG AGC CAC CAG AGG | — | 14 |
| 188 Gly188Glu | | GTC CCC TGG GCT CGC- 3′ | | |
| G/A | StrM16A | 5′ -GCG AGC CAC CAG AGA | — | 15 |
| | | GTC CCC TGG GCT CGC- 3′ | | |
| LIPC/ | StrM6C | 5′ -GCG AGC TTT TGA CAG GGG | — | 16 |
| 480 C-480T C/T | | GTG AAG G GCT CGC- 3′ | | |
| | StrM6T | 5′ -GCG AGC TTT TGA CAG GGG | — | 17 |
| | | GTG AAG G GCT CGC-3′ | | |
| CETP IV/ | StrM19G | 5′ -GCG AGC CCG AGT CCG | M19 target: | 18 |
| WIAF-10949 | | TCC AGA GCT GCT CGC- 3′ | perfect match | |
| G/A | StrM19A | 5′ -GCG AGC CCG AGT CCA | M19 target: | 19 |
| | | TCC AGA GCT GCT CGC- 3′ | one C/A | |
| | | | mismatch | |
| TCF1/ | StrNCb | See Table I | 142 CC target: | 1 |
| 142 | | | perfect match | |
| | StrNTb | See Table I | 142 CC target: | 2 |
| | | | one T/G | |
| | | | mismatch | |
| | StrNGb | See Table I | 142 CC target: | 3 |
| | | | one G/G | |
| | | | mismatch | |
| | StrNAb | See Table I | 142 CC target: | 4 |
| | | | one A/G | |
| | | | mismatch | |
| | StrNC | 5′ -GCG AGC CAA CCA CTC | 142 CC target: | 20 |
| | | CAC CTG TC GCT CGC- 3′ | one T/C | |
| | | | mismatch | |
| | StrNT | 5′ -GCG AGC CAA CCA TTC CAC | 142 CC target: | 21 |
| | | CTG TC GCT CGC- 3′ | two T/G and | |
| | | | T/C | |
| | | | mismatches | |
| | StrNG | 5′0 -GCG AGC CAA CCA GTC | 142 CC target: | 22 |
| | | CAC CTG TC GCT CGC- 3′ | two G/G and | |
| | | | T/C | |
| | | | mismatches | |
| | StrNA | 5′ -GCG AGC CAA CCA ATC | 142 CC target: | 23 |
| | | CAC CTG TC GCT CGC- 3′ | two G/G and | |
| | | | T/C | |
| | | | mismatches | |

Examples of Hairpin Probes Design

Probes shown in table III were designed to analyse mutations occurring in individuals affected by Charcot-Marie-Tooth disease.

Charcot-Marie-Tooth disease is the most frequent hereditary disease of the peripheral nervous system. Heterogeneous in essence, (multigenic disease) it is characterised by a cohort of sensory and motricity neuropathic disorders (http://molgen-www.uia.ac.be/CMTMutations/CMT.cfm).

At the molecular level, 14 genes are now known to be responsible of apparition of Charcot-Marie-Tooth neuropathy through their mutations. Among them, peripheral myelin protein gene (PMP22), myelin protein zero gene (MPZ), and connexion 32 gene (GJB1) which mutations are dominant, are used in this example to design probes for analysis of 5 mutations listed thereafter:

| Mutation Name | Type | Gene |
|---|---|---|
| C42R T > C | Substitution T > C | PMP22 |
| W140R T > C | Substitution T > C | PMP22 |
| T124M C > T | Substitution C > T | MPZ |
| V113F G > T | Substitution G > T | MPZ |
| S26L C > T | Substitution C > T | GJB1 |

Probe design was done according to steps described in probe design section in the detailed description of the invention.

These steps are:
1—Designing the loop and stem sequence of mutant and wild type probes, and calculating perfect and imperfect hybrids Tms with Meltcalc
2—Adding probes arms (stems), checking structure and calculating Tm of hairpin probes with Mfold.
3—Alignment of the probes with their respective targets, and verification of lack of homology between hairpin probes and non-complementary targets.

All probes designed for analysing these mutations are listed in table III. These probes were used to design a biochip, by using the following procedure:

Each probe was synthesised with an aminolinker in 5′ (Sigma Genosys, UK). These probes were spotted on activated glass slides (Genescore, France) using a Microgrid II Biorobotics spotter. Upon spotting, a covalent link is formed between glass substrate and aminated end of probe. Five replicates of each spot were done.

Such biochips were hybridised with a set of oligonucleotide targets (Table IV) fully complementary with probes of table III, and 5′ labelled with a fluorescent dye (Cyanin 3, Sigma Genosys).

TABLE III

| | | SEQ |
|---|---|---|
| | | ID |
| Hairpin probes | 5′-3′ | No |

| | | |
|---|---|---|
| Neu1S26LW6 | GCGAGCAGTATGGCTCTCGGTCATGCTCGC | 24 |
| Neu1S26LM6 | GCGAGCAGTATGGCTCTTGGTCATGCTCGC | 25 |
| Neu4V113FW2 | GCGAGCGCTCCATTGTCATACACAAGCTCGC | 26 |
| Neu4V113FM2 | GCGAGCGCTCCATTTTCATACACAAGCTCGC | 27 |
| Neu4T124MW3 | GCGAGCCAATGGCACGTTCACTTGCTCGC | 28 |
| Neu5C42RW1 | GCGAGCCAGAACTGTAGCACCGCTCGC | 29 |
| Neu5C42RM1 | GCGAGCCAGAACCGTAGCACCGCTCGC | 30 |
| Neu6W140RW4 | GCGACGTCCTGGCCTGGGTGCGTCGC | 31 |
| Neu6W140RM4 | GCGACGTCCTGGCCCGGGTGCGTCGC | 32 |

Two sets of experiments were performed on two different biochips. The first chip was hybridised with all targets corresponding to wild type alleles (WT, table IV(a)). The second chip was hybridised with all targets corresponding to mutant alleles (MT, table IV(a)).

TABLE IV (a)

| WT | | |
|---|---|---|
| targets | 5′-3′ | SEQ ID No |

| | | |
|---|---|---|
| CiS26LW | ATGAAGATGACCGAGAGCCATACTCGGCCA | 33 |
| CiV113FW | TCTAGGTTGTGTATGACAATGGAGCCATCC | 34 |
| CiT124MW | CGTCACAAGTGAACGTGCCATTGTCACTGT | 35 |
| CiC42RW | GAAGAGGTGCTACAGTTCTGCCAGAG | 36 |
| CiW140RW | GAAGGCCACCCAGGCCAGGATGTAGG | 37 |

TABLE IV (b)

| MT | | |
|---|---|---|
| targets | 5′-3′ | SEQ ID No |

| | | |
|---|---|---|
| CiS26LM | ATGAAGATGACCAAGAGCCATACTCGGCCA | 38 |
| CiV113FM | TCTAGGTTGTGTATGAAAATGGAGCCATCC | 39 |
| CiC42RM | GAAGAGGTGCTACGGTTCTGCCAGAG | 40 |
| CiW140RM | GAAGGCCACCCGGGCCAGGATGTAGG | 41 |

For each of these hybridisations, conditions were set up at: 100 nM targets in 6×SSC buffer, 1M NaCl, hybridised for 1 h at room temperature, 20 μL of target per biochip. Mean value for 5 replicates, scanning on GSI lumonics scanner, laser and PMT at 500×500.

Fluorescent median intensities of each wild type and mutant probes were analysed after scanning, and used to calculate the discrimination ratio of each probe (Rd).

The discrimination ratio of probes (Rd) is defined as:
Rd=fluorescence intensity for perfect hybrid/fluorescence intensity for imperfect hybrid.

With fluorescence intensity for perfect hybrid=the intensity measured upon hybridisation of target fully complementary with considered hairpin probe (e.g. hybridisation of the target CiS26LW with hairpin probe Neu1S26LW6, or hybridisation of the target CiS26LM with hairpin probe Neu1S26LM6) and fluorescence intensity for im perfect hybrid=the intensity measured upon hybridisation of target with one mismatch (e.g. hybridisation of the target CiS26LW with hairpin probe Neu1S26LM6, or hybridisation of the target CiS26LM with hairpin probe Neu1S26LW6).

The discrimination ratio (Rd) has the following meaning:

For example, a Rd of 10 for a given hairpin probe, means that hybridisation of a target fully complementary with his hairpin will generate a signal 10 times more important than hybridization with a target possessing one base difference with first target.

These Rd values are reported in table V, as well as calculated Tm value of loops and stems of each of the hairpin probes.

The Dtm parameter is also calculated for each of these probes, with Dtm=Tm stem-Tm loop. This parameter describes the “relative power” of discrimination of probes. The higher the Dtm is (stem Tm high and loop Tm weak), the higher is the capability to discriminate two sequences of high homology.

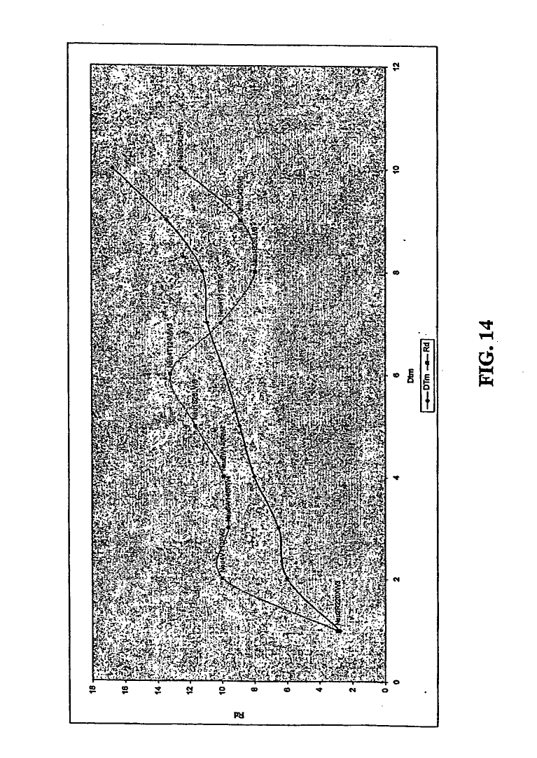

All these values were plotted in a graph (FIG. 14) showing relationship between evolution of Dtm and discrimination ratio Rd.

TABLE V

| Name | Loop Tm | Stem Tm | DTm | Rd |
|---|---|---|---|---|
| Neu4V113FW2 | 63.1 | 69.1 | 6 | 9.95 |
| Neu6W140RW4 | 67.3 | 73.8 | 6.5 | 9.61 |
| Neu6W140RM4 | 63.6 | 71.6 | 8 | 9.9 |
| Neu1S26LW6 | 65 | 73.9 | 8.9 | 11.65 |
| Neu4T124MW3 | 62.8 | 72.6 | 9.8 | 13.16 |
| Neu4V113FM2 | 61.1 | 72 | 11 | 10.04 |
| Neu1S26LM6 | 62.7 | 73.9 | 11 | 7.93 |
| Neu5C42RM1 | 60 | 73.4 | 13 | 8.76 |
| Neu5C42RW1 | 56.7 | 73.4 | 17 | 12.48 |

This relation between Rd and Dtm may be used to select loops and stems in the design of probes, having suitable composition and length for the desired analysis. For example, if a Rd of 10 is required for design of all probes, Dtm value will be selected to be 9.

Example of Mutation Analysis Result on Human Genomic DNA Sample

This example is specifically directed to the analysis of wild type allele of W140R mutation of exon 3 of gene PMP22, one of the mutations responsible for Charcot-Marie-Tooth disease.

All hairpin probes which sequence is given in table III is immobilised on a biochip.

Genomic DNA of exon3 of PMP22 gene was PCR amplified using a pair of primers, one being 5′ end labelled with a fluorescent cyanin 3 molecule.

PCR was performed as following: 100 ng of DNA from healthy individual were used with a PCR kit (High fidelity expand PCR, Roche) using manufacturer protocol and specific PCR primers for 40 cycles. A negative standard (PCR well lacking genomic DNA) was included as well during PCR.

PCR products were controlled by electrophoresis on an agarose gel to verify PCR product length, and lack of contamination for the negative standard. Amplified DNA was quantified by UV adsorption at 260 nm after purification on microcolumn (Quiaquick, Qiagen).

1.4 μg of Cy3-labelled DNA from PCR of exon3 of gene PMP22 was hybridised for 1 hour at room temperature on biochip of hairpin probes.

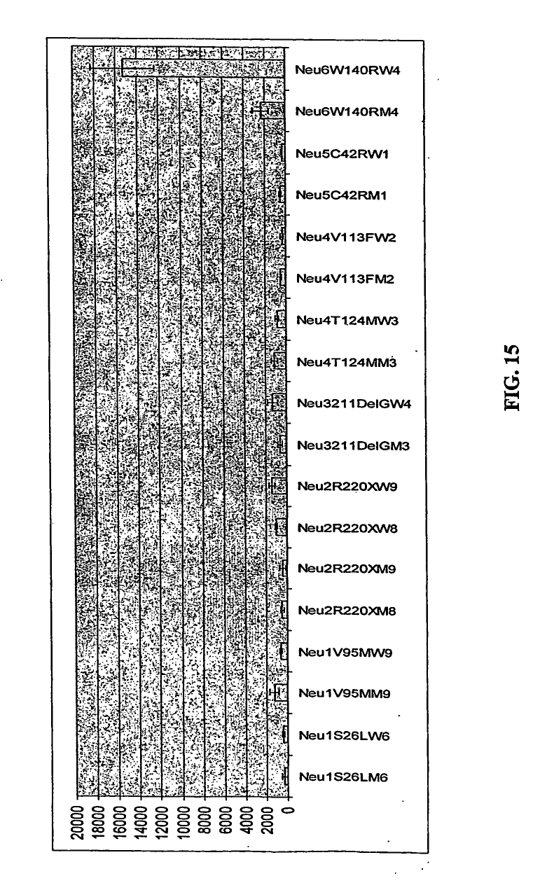

Following hybridisation, the biochip was scanned and results were reported in FIG. 15.

These results show that PCR products are mainly hybridised on complementary probes, or probes having one base difference (probes Neu6W140RM4, and Neu6W140RM4). Rd is 6,61 (WT/MT probes signal).

The given genotype for this analysis is wild type homozygote for W140R mutation, which corresponds to “healthy” phenotype of tested individual.

### REFERENCES CITED

Citation of a reference herein shall not be construed as an admission that such reference is prior art to the present invention.

Many modifications and variations of the present invention can be made without departing from its spirit and scope, as will be apparent to those skilled in the art.

1-45. (canceled)

46. An unlabelled probe comprising:
(a) a target-specific sequence that is from 6 to 30 nucleotides in length;
(b) a first arm that is less than 10 nucleotides in length and is 5′ of the target specific sequence; and
(c) a second arm that is less than 10 nucleotides in length and is 3′ of the target specific sequence,
the target-specific sequence being not complementary with any other portion of the unlabelled probe; and the first arm and the second arm being perfectly complementary to each other.

47. The probe of claim 46, wherein the target-specific sequence is from 10 to 25 nucleotides in length.

48. The probe of claim 47, wherein the target-specific sequence is from 15 to 20 nucleotides in length.

49. The probe of claim 46, wherein, when a target molecule is hybridized with the target-specific sequence, said probe adopts an “open” conformation, and a “reporter” molecule can hybridize to the first arm or the second arm.

50. The probe of claim 49, wherein the “reporter” molecule comprises less than 10 nucleotides which are perfectly complementary with the nucleic acid sequence of the first arm or the second arm.

51. The unlabelled probe of claim 49, wherein the “reporter” molecule comprises a detectable marker.

52. The probe of claim 51, wherein the detectable marker is a nucleotide analog, a fluorescent label, biotin, imminobiotin, an antigen, a cofactor, dinitrophenol, lipoic acid, an olefinic compound, a polypeptide, an electron-rich molecule, an enzyme, or a radioactive isotope.

53. The probe of claim 46, wherein a Dtm (the difference between melting temperature (Tm) of perfect hybrid formed upon association of the target-specific sequence with the target molecule and melting temperature (Tm) of perfect hybrid formed by association of the first arm and the second arm) is greater than 10.

54. The probe of claim 53, wherein the Dtm is equal to 15.

55. The probe of claim 46, wherein a Dtm (the difference between melting temperature (Tm) of perfect hybrid formed upon association of the target-specific sequence with the target molecule and melting temperature (Tm) of perfect hybrid formed by association of the first arm and the second arm) is lower than 10.

56. A probe biochip comprising a substrate; and at least two probes of claim 46.

57. The probe biochip of claim 56, wherein the probe is attached to substrate.

58. The probe biochip of claim 57, wherein said probe further comprise a linker, and is attached to the substrate by mean of said linker.

59. The probe biochip of claim 57, wherein the substrate consists of a functionalized glass surface, a functionalized plastic surface, a functionalized metal, a conductive metal surface, a conductive plastic surface, a porous substrate, a porous metal, an optical fiber, a glass fiber derived substrate, silicon dioxide, a functional lipidic membrane, a liposome, or a filtration membrane.

60. The probe biochip of claim 56, wherein all of the first arms have an identical sequence.

61. The probe biochip of claim 60, wherein one reporter molecule can hybridize with each probe.

62. The probe biochip of claim 56, wherein all perfect hybrids formed upon association of target-specific sequences with the target molecules have a melting temperature equal within a range of 4° C.

63. The probe biochip of claim 62, wherein all perfect hybrids formed upon association of target-specific sequences with the target molecules have a melting temperature equal within a range of 1° C.

64. The probe biochip of claim 56, wherein a difference between melting temperature of hybrid formed upon association of the target-specific sequence with the target molecule, and melting temperature of hybrid formed upon association of the target-specific sequence with a molecule for which the target specific sequence is not designed is greater or equal to 5° C.

65. The probe biochip of claim 64, wherein said difference between melting temperature is greater or equal to 8° C.

66. The probe biochip of claim 56, wherein a Dtm of at least two probes (the difference between melting temperature (Tm) of perfect hybrid formed upon association of the target-specific sequence with the target molecule and melting temperature (Tm) of perfect hybrid formed by association of the first arm and the second arm) are equal within a range of 1° C.

67. An unlabelled universal addressing system comprising:
(a) at least two unlabelled and non-immobilised first probes comprising (i) a target-specific sequence that is 6 to 30 nucleotides in length; and (ii) a tag sequence connected to the 5′ or 3′ end of said target specific sequence; and,
(b) a biochip comprising a substrate and at least two second immobilised probes of claim 11;
each first probe's tag sequence being different for each probe and complementary with the target-specific sequence of one of the second probes.

68. The unlabelled universal addressing system of claim 67, wherein said first unlabelled probes comprises:
(i) a target-specific sequence that is 6 to 30 nucleotides in length;
(ii) a first arm that is less than 10 nucleotides in length and is 5′ of said target-specific sequence;
(iii) a second arm that is less than 10 nucleotides in length and is 3′ of said target-specific sequence;
(iv) a tag sequence that is from 10 to 50 nucleotides in length, connected to the first arm or the second arm.
the target-specific sequence being not complementary with any other portion of said unlabelled probe; and the first arm and the second arm being perfectly complementary to each other.

69. The universal addressing system of claim 68, wherein all of the first arms of first probes have an identical sequence.

70. The universal addressing system of claim 67, wherein all of the first arms of second probes have an identical sequence.

71. The universal addressing system of claim 67, wherein a same reporter molecule can hybridize to the first probe or the second probes.

72. A kit comprising the biochip of claim 56 and one or more reagents.

73. The kit of claim 72, further comprising a set of non-immobilized probes of claim 46.

74. The kit of claim 72, further comprising a reporter molecule.

75. A method of nucleic acid detection comprising:
(a) contacting ex vivo a nucleic acid sample with a biochip of claim 56 or with an universal addressing system of claim 67; and
(b) detecting a signal from at least one probe of the biochip or universal addressing system which has assumed an open conformation following contacting in step (a).

76. A method of detecting a genetic variant ex vivo in a nucleic acid sample comprising:
(a) contacting the sample with a biochip of claim 56 or with an universal addressing system of claim 67, wherein at least one probe of the biochip or the universal addressing system is a probe specific of the genetic variant, and
(b) detecting a signal from the probe specific of the genetic variant, the signal detected in step (b) indicating the presence of the genetic variant in nucleic acid sample.

77. The method of claim 76, wherein the detected genetic variant is a single nucleotide polymorphism.

78. A method of detecting ex vivo any nucleic acid containing organism or a remnant thereof comprising:
(a) contacting the nucleic acid sample with a biochip of claim 56 or with an universal addressing system of claim 67, wherein at least one of said probe is specific for a nucleic acid of the organism or a remnant thereof; and
(b) detecting a signal from the probe specific for a nucleic acid of the organism, the signal detected in step (b) indicating the presence of the nucleic acid containing organism or a remnant thereof.

79. The method of claim 78, wherein the nucleic acid containing organism is a virus or a bacterium.

80. A method of detecting ex vivo an alternative splice product of a gene in a nucleic acid sample comprising:
(a) contacting the sample with a biochip of claim 56 or with an universal addressing system of claim 67, wherein at least one probe of the biochip or the universal addressing system is a hairpin probe specific for an exon of the gene or specific for a junction of two exons; and
(b) detecting a signal from the specific for an exon of the gene or specific for a junction of two exons, the signal detected in step (b) indicating the presence of the alternative splice product of the gene in the nucleic acid sample.

81. The method of claim 80, wherein the nucleic acid sample comprises mRNA, or cDNA.

82. A method of ex vivo sequencing an oligonucleotide comprising:
(a) contacting the sample containing the oligonucleotide with a biochip of claim 56 or with an universal addressing system of claim 67; and
(b) detecting a signal from at least one probe of the biochip;
the signal detected in step (b) being used for determining the sequence of the oligonucleotide.

83. A method of detecting allelic imbalances and loss of heterozygosity ex vivo in a nucleic acid sample comprising:
(a) amplifying of at least one chromosomal DNA region of microsatellite type, using a pair of primers from at least two nucleic acid samples from biological fluids or tissues, wherein at least one of the samples from fluids or tissues is having no allelic imbalance or loss of heterozygosity, and each tissue or fluid is differentially labeled during amplification;
(b) eliminating of said primers after amplification;
(c) contacting of said amplification products with a biochip of claim 56 or with an universal addressing system of claim 72, wherein at least one probe of the biochip or the universal addressing system is complementary to a primer used for amplifying said chromosomal DNA region; and
(d) detecting the signals from at least one probe of said biochip,
the signals detected in step (d) being used to determine the presence of an allelic imbalance or loss of heterozygosity in one of the nucleic acid samples.


SEQUENCE LISTING (41 sequences)

SEQ ID NO: 1 - DNA, 30 bp, Artificial Sequence
  Description of Artificial Sequencehairpin probe
gcgagccaaccagtcccacctgtcgctcgc

SEQ ID NO: 2 - DNA, 30 bp, Artificial Sequence
  Description of Artificial Sequence hairpin probe
gcgagccaaccagttccacctgtcgctcgc

SEQ ID NO: 3 - DNA, 30 bp, Artificial Sequence
  Description of Artificial Sequence hairpin probe
gcgagccaaccagtgccacctgtcgctcgc

SEQ ID NO: 4 - DNA, 30 bp, Artificial Sequence
  Description of Artificial Sequence hairpin probe
gcgagccaaccagtaccacctgtcgctcgc

SEQ ID NO: 5 - DNA, 42 bp, Artificial Sequence
  Description of Artificial Sequence oligonucleotide target
aggtgttgggacaggtgggactggttgaggccagtggtatcg

SEQ ID NO: 6 - DNA, 42 bp, Artificial Sequence
  Description of Artificial Sequence oligonucleotide target
aggtgttgggacaggtggaactggttgaggccagtggtatcg

SEQ ID NO: 7 - DNA, 120 bp, Homo sapiens
atcaagggttacatgcagcaacacaacatcccccagagggaggtggtcgatgtcaccggcctgaaccagtcgcacctctcccagcatctcaacaagggcacccctatgaagacccagaag

SEQ ID NO: 8 - DNA, 50 bp, Homo sapiens
atcaagggttacatgcagcaacacaacatcccccaggacccagaagcgtg

SEQ ID NO: 9 - DNA, 24 bp, Artificial Sequence
  Description of Artificial Sequence oligonucleotide target
cacgcttctgggtcctgggggatg

SEQ ID NO: 10 - DNA, 30 bp, Artificial Sequence
  Description of Artificial Sequence hairpin probe
gcgagccccccaggacccagaagcgctcgc

SEQ ID NO: 11 - DNA, 6 bp, Artificial Sequence
  Description of Artificial Sequence probe
gcgagc

SEQ ID NO: 12 - DNA, 35 bp, Artificial Sequence
  Description of Artificial Sequence hairpin probe
gcgagcgaataagaagtaggctggtgagcgctcgc

SEQ ID NO: 13 - DNA, 35 bp, Artificial Sequence; identical to SEQ ID NO: 12
  Description of Artificial Sequence hairpin probe

SEQ ID NO: 14 - DNA, 30 bp, Artificial Sequence
  Description of Artificial Sequence  hairpin probe
gcgagccaccagagggtcccctgggctcgc

SEQ ID NO: 15 - DNA, 30 bp, Artificial Sequence
  Description of Artificial Sequence hairpin probe
gcgagccaccagagagtcccctgggctcgc

SEQ ID NO: 16 - DNA, 31 bp, Artificial Sequence
  Description of Artificial Sequence hairpin probe
gcgagcttttgacagggggtgaagggctcgc

SEQ ID NO: 17 - DNA, 31 bp, Artificial Sequence; identical to SEQ ID NO: 16
  Description of Artificial Sequence hairpin probe

SEQ ID NO: 18 - DNA, 30 bp, Artificial Sequence
  Description of Artificial Sequence hairpin probe
gcgagcccgagtccgtccagagctgctcgc

SEQ ID NO: 19 - DNA, 30 bp, Artificial Sequence
  Description of Artificial Sequence hairpin probe
gcgagcccgagtccatccagagctgctcgc

SEQ ID NO: 20 - DNA, 29 bp, Artificial Sequence
  Description of Artificial Sequence hairpin probe
gcgagccaaccactccacctgtcgctcgc

SEQ ID NO: 21 - DNA, 29 bp, Artificial Sequence
  Description of Artificial Sequence hairpin probe
gcgagccaaccattccacctgtcgctcgc

SEQ ID NO: 22 - DNA, 29 bp, Artificial Sequence
  Description of Artificial Sequence hairpin probe
gcgagccaaccagtccacctgtcgctcgc

SEQ ID NO: 23 - DNA, 29 bp, Artificial Sequence
  Description of Artificial Sequence hairpin probe
gcgagccaaccaatccacctgtcgctcgc

SEQ ID NO: 24 - DNA, 30 bp, Artificial sequence
  Description of Artificial Sequence hairpin probe
gcgagcagtatggctctcggtcatgctcgc

SEQ ID NO: 25 - DNA, 30 bp, artificial sequence
  Description of Artificial Sequence hairpin probe
gcgagcagtatggctcttggtcatgctcgc

SEQ ID NO: 26 - DNA, 31 bp, artificial sequence
  Description of Artificial Sequence  hairpin probe
gcgagcgctccattgtcatacacaagctcgc

SEQ ID NO: 27 - DNA, 31 bp, artificial sequence
  Description of Artificial Sequence hairpin probe
gcgagcgctccattttcatacacaagctcgc

SEQ ID NO: 28 - DNA, 29 bp, artificial sequence
  Description of Artificial Sequence hairpin probe
gcgagccaatggcacgttcacttgctcgc

SEQ ID NO: 29 - DNA, 27 bp, artificial sequence
  Description of Artificial Sequence hairpin probe
gcgagccagaactgtagcaccgctcgc

SEQ ID NO: 30 - DNA, 27 bp, Artificial sequence
  Description of Artificial Sequence hairpin probe
gcgagccagaaccgtagcaccgctcgc

SEQ ID NO: 31 - DNA, 26 bp, artificial sequence
  Description of Artificial Sequence hairpin probe
gcgacgtcctggcctgggtgcgtcgc

SEQ ID NO: 32 - DNA, 26 bp, artificial sequence
  Description of Artificial Sequence hairpin probe
gcgacgtcctggcccgggtgcgtcgc

SEQ ID NO: 33 - DNA, 30 bp, artificial sequence
  Description of Artificial Sequence oligonucleotide target
atgaagatgaccgagagccatactcggcca

SEQ ID NO: 34 - DNA, 30 bp, artificial sequence
  Description of Artificial Sequence oligonucleotide target
tctaggttgtgtatgacaatggagccatcc

SEQ ID NO: 35 - DNA, 30 bp, artificial sequence
  Description of Artificial Sequence oligonucleotide target
cgtcacaagtgaacgtgccattgtcactgt

SEQ ID NO: 36 - DNA, 26 bp, artificial sequence
  Description of Artificial Sequence oligonucleotide target
gaagaggtgctacagttctgccagag

SEQ ID NO: 37 - DNA, 26 bp, artificial sequence
  Description of Artificial Sequence oligonucleotide target
gaaggccacccaggccaggatgtagg

SEQ ID NO: 38 - DNA, 30 bp, artificial sequence
  Description of Artificial Sequence oligonucleotide target
atgaagatgaccaagagccatactcggcca

SEQ ID NO: 39 - DNA, 30 bp, artificial sequence
  Description of Artificial Sequence oligonucleotide target
tctaggttgtgtatgaaaatggagccatcc

SEQ ID NO: 40 - DNA, 26 bp, artificial sequence
  Description of Artificial Sequence oligonucleotide target
gaagaggtgctacggttctgccagag

SEQ ID NO: 41 - DNA, 26 bp, artificial sequence
  Description of Artificial Sequence oligonucleotide target
gaaggccacccgggccaggatgtagg